(19) United States
(12) Patent Application Publication
(10) Pub. No.: US 2025/0204813 A1
(43) Pub. Date: Jun. 26, 2025

(54) SYSTEMS AND METHODS FOR MANAGING CHRONIC DISEASE USING ANALYTE AND PATIENT DATA

(71) Applicant: Intuity Medical, Inc., Fremont, CA (US)

(72) Inventors: Emory V. ANDERSON III, Danville, CA (US); Robin Susanne Gaffney, Redwood City, CA (US); Michael F. Tomasco, Morgan Hill, CA (US); Raúl Escutia, Sunnyvale, CA (US); Paul D. Reynolds, Palo Alto, CA (US)

(21) Appl. No.: 18/786,318

(22) Filed: Jul. 26, 2024

Related U.S. Application Data
(63) Continuation of application No. 15/953,295, filed on Apr. 13, 2018, now abandoned.
(60) Provisional application No. 62/485,362, filed on Apr. 13, 2017.

(51) Int. Cl. A61B 5/145; A61B 5/00; A61B 5/021; A61B 5/024; A61B 5/11; G01C 22/00; G06Q 10/1093; G09B 19/00; G16H 20/30; G16H 20/60; G16H 20/70; G16H 40/63; G16H 40/67; G16H 50/30; G16H 80/00

(52) U.S. Cl. CPC A61B 5/14532; A61B 5/0002; A61B 5/021; A61B 5/024; A61B 5/1118; A61B 5/4812; A61B 5/4815; A61B 5/4842; A61B 5/681; A61B 5/6898; A61B 5/7275; A61B 5/743; A61B 5/744; A61B 5/746; G06Q 10/1095; G09B 19/00; G09B 19/0092; G16H 40/63; G16H 40/67; G16H 50/30; G16H 80/00; A61B 2562/0219; G01C 22/006; G16H 20/30; G16H 20/60; G16H 20/70

(57) ABSTRACT

Devices, systems, and methods herein relate to managing a chronic condition such as diabetes. These systems and methods may obtain patient data from a plurality of devices, integrate the data for analysis of trends that may be presented to the patient and/or health care professional along with an actionable suggestion. In some variations, a method may include the steps of receiving analyte data generated by an analyte measurement device and patient data generated by a patient measurement device. One or more data trends may be generated by analyzing the analyte data against the patient data using a computing device. The device settings of one or more of the analyte measurement device and the computing device may be modified in response to one or more of the data trends.

### CROSS-REFERENCE TO RELATED APPLICATIONS

This application is a continuation of U.S. patent application Ser. No. 15/953,295, filed Apr. 13, 2018, which claims priority to U.S. Provisional Application Ser. No. 62/485,362, filed on Apr. 13, 2017, the contents of each of which is hereby incorporated by reference in its entirety.

### FIELD

Devices, systems, and methods herein relate to measuring an analyte in a fluid sample (e.g., blood glucose) that may be used in managing a chronic disease, including but not limited to diabetes.

### BACKGROUND

Diabetes is a widespread condition, affecting millions worldwide. In the United States alone, over 20 million people are estimated to have the condition. Diabetes accounts for an estimated $174 billion annually in direct and indirect medical costs. Depending on the type (Type 1, Type 2, and the like), diabetes may be associated with one or more symptoms such as fatigue, blurred vision, and unexplained weight loss, and may further be associated with one or more complications such as hypoglycemia, hyperglycemia, ketoacidosis, neuropathy, and nephropathy.

To help delay or prevent these undesirable complications, it may be helpful for people with diabetes to monitor one or more blood analyte levels, such as blood glucose. Glucose testing allows a patient to ensure that his or her blood glucose is at a safe level, which in turn may help monitor the effectiveness of diet, medication, and exercise in controlling the patient's diabetes, and may also help reduce the risk of developing one or more diabetes-related conditions (e.g., blindness, kidney damage, and nerve damage). However, glucose testing data gathered from many of the currently available glucose meters may be difficult for many patients to interpret and understand, and may result in a frustrating or otherwise negative patient experience (which may reduce the likelihood of patient compliance). As such, it may be desirable to provide a patient with one or more actionable suggestions with respect to their behavior and operation of the devices used to manage their diabetes.

### SUMMARY

Described here are analyte measurement devices and disease management systems and methods for providing an actionable suggestion to a patient and/or a health care professional in managing a chronic condition such as diabetes. These systems and methods may, for example, obtain patient data from a plurality of devices, integrate the data, and analyze it for trends that may be presented to the patient and/or health care professional along with one or more suggestions that the patient and/or health care professional may take action on in view of the trends. This may, for example, give the patient further insight into their condition and provide a tangible step to improving their health. For some patients, review of their data and trends may be burdensome. However, the systems and methods described herein may provide a summary of the data and trends to the patient to allow them to more easily monitor their condition. The suggestion may include one or more steps that the patient, the patient's device, and/or the patient's support network (e.g., health care professional, coach, partner, family, friends) may perform to help a patient manage his or her diabetes in view of the data trends. In some variations, data from a plurality of patients may be analyzed for trends, allowing patients with similar characteristics to be grouped together. Patients may be prompted to join a support group of similar patients, as described in more detail herein. A health care professional may prioritize patient care using these sets of similar patients. The health care professional may, in some variations, be presented with an actionable suggestion and/or observation that they may execute for groups of similar patients, thus potentially increasing efficiency and reducing costs.

Generally, the systems and methods described herein may receive data from an analyte measurement device (e.g., blood glucose monitor) and a patient measurement device (e.g., activity tracker) that measures one or more patient health characteristics. The data generated from each of these devices may be automatically uploaded to a patient's computing device (e.g., smartphone, laptop, PC) and/or a database (e.g., cloud based storage). The data may be integrated so as to permit analysis for trends between the analyte data and the other patient data for one or more patients. These trends may be presented to the patient on any device (e.g., analyte measurement device, smartphone, laptop) with selectable levels of complexity (e.g., detailed, concise, summary, long-term, short-term). Presenting the trends to the patient in an easily accessible and customizable manner may increase the patient's understanding of how their behavior (e.g., exercise, diet, testing, and medication compliance) correlates with their blood glucose measurements. A health care professional may also be granted access to the trend data of one or more patients.

Furthermore, the systems and methods may generate an actionable suggestion in response to the trends. The suggestion may include an action that the patient may perform themselves (e.g., go for a walk in the afternoon, cat carbohydrates) and/or an action to be performed by a patient device (e.g., set a testing reminder, present a health article, video, podcast). The actionable suggestion may be output on a patient device as a prompt that the patient may select to confirm execution of the suggested action. In some variations, the suggested action may be executed automatically without user input and the prompt output to the patient may notify the patient of the action taken (e.g., call placed to health care professional, text message sent to family member). In some variations, the actionable suggestion may be based on observations derived from the analyte data, patient data, and trends.

In some variations, a method of monitoring a chronic condition of a patient may include the steps of receiving analyte data generated by an analyte measurement device and patient data generated by a patient measurement device. One or more data trends may be generated by analyzing the analyte data against the patient data using a computing device comprising a processor and memory. The device settings of one or more of the analyte measurement device and the computing device may be modified in response to one or more of the data trends.

In some variations, the method may further include the steps of transferring the analyte data from the analyte measurement device to the computing device at predetermined intervals. In some variations, one or more of the data trends may be output using the computing device.

In some variations, at least one prompt to modify patient behavior may be output in response to one or more of the data trends. In some of these variations, the prompt may comprise encouragement to comply with one or more of a testing, diet, and exercise regimen. In some variations, at least one prompt may be output to modify the device settings in response to one or more of the data trends. In some of these variations, a set of prompts may be output to modify the device settings at predetermined intervals. In some variations, modifying the device settings includes modifying one or more of frequency, timing, and content of patient notification.

In some variations, a set of one or more predetermined contacts may be notified based on a characteristic of the one or more of the data trends. In some variations, a set of predetermined contacts of the patient's condition may be notified in response to one or more of the data trends being a high risk condition. In some variations, a set of predetermined contacts may be notified of the patient's condition in response to one or more of the data trends being an improving health condition. In some variations, the set of predetermined contacts may comprise one or more of a health care professional, a patient's partner, family member, and support group. In some variations, health care professional attention may be determined to be urgent in response to one or more of the data trends being a high risk condition. In some variations, an appointment between the patient and a health care professional may be scheduled using the computing device in response to one or more of the data trends being a high risk condition. In some variations, at least one prompt may be output to modify health care professional device settings in response to one or more of the data trends.

In some variations, a communication channel may be established between the computing device and a health care professional device in response to one or more of the data trends being a high risk condition. In some of these variations, the analyte data, the patient data, and the one or more data trends may be received at the health care professional device, and at least one prompt may be output to modify patient behavior and the device settings at the health care provider device. In other of these variations, a prompt may be transmitted comprising a suggestion from the health care professional device to the computing device using the communication channel. In some variations, the analyte measurement device may include a blood glucose monitor and the patient measurement device may include one or more of an activity tracker, a heart rate monitor, a blood pressure monitor, a scale, an A1c monitor, and a cholesterol monitor. In some variations, the analyte data may comprise blood glucose data and blood glucose testing history. In some variations, the patient data may comprise one or more of activity data, nutrition data, drug data, hydration data, sleep data, blood pressure data, heart rate data, cholesterol data, A1c data, weight data, geolocation data, mental health data, and patient data. In some variations, one or more data trends generated may comprise one or more of performing time synchronization and range normalization of the analyte data and the patient data.

In some variations, one or more of the generated data trends may comprise generating a wellness indicator based at least in part on the analyte data and the patient data. In some of these variations, the wellness indicator is governed by the equation:

$$Ws = 100 - a ( s \cdot d ( glucose over 30 days ) ) - b ( \vert target glucose - avg ( glucose over 30 days ) \vert ) - c ( number of hypoglycemic readings over 30 days ) - d ( number of hyperglycemic readings over 30 days ) + e ( \% of readings in target range - 60 \% ) + f ( number of glucose measurements over 30 days ) + g ( \frac{minutes of activity over previous 7 days}{60} ) - h ( minutes of activity - target minutes of activity ) - i ( grams of carbohydrates consumed over previous day ) - j ( \begin{matrix}grams of carbohydrates consumed over previous day \\ above target grams of carbohydrates consumed over previous day\end{matrix} ) - k ( BMI ) + l ( number of meals marked ) + m ( \frac{number of hours of sleep over 7 days}{7} ) + n ( number of doctor visits over previous 365 days ) + p ( number of eye exams over previous 365 days ) + q ( number of diabetic foot exams over previous 365 days ) ,$$

where a, b, c, d, e, f, g, h, i, j, k, l, m, n, p, and q are scale factors, s.d. is standard deviation, and BMI is Body Mass Index.

In some variations, a high risk condition may be determined based on a comparison between at least one of blood glucose data of the analyte data and activity data of the patient data relative to at least one predetermined threshold. In some variations, generating the one or more data trends may comprise estimating a risk of a hypoglycemic event based at least in part on the analyte data and/or the patient data, wherein the analyte data comprises blood glucose data and the patient data comprises one or more of activity data and nutrition data. In some of these variations, the risk of the hypoglycemic event is governed by the equation:

$$\frac{( \frac{AvgGlu}{Current Glucose} ) * ( Act * Exe ) - ( Carbs * 4 )}{100} ,$$

where AvgGlu is an average blood glucose value over the 90 preceding days, Current Glucose is a current blood glucose value, Act is a number of minutes of patient activity over a predetermined time interval, Exe is an exertion level based on heart rate, and Carbs is a number of grams of carbohydrates consumed in the 90 preceding minutes. In other of these variations, the risk of the hypoglycemic event is governed by the equations: Glu<150 mg/DL and Act*Exe>200, where Glu is a current glucose value, Act is a number of minutes of patient activity over a predetermined time interval, and Exe is an exertion level based on heart rate.

In some other variations, a method of managing a patient population may include the steps of receiving analyte data generated by a plurality of analyte measurement devices and patient data generated by a plurality of patient measurement devices. The analyte data and the patient data may correspond to a plurality of patients. One or more data trends may be generated by analyzing the analyte data against the patient data for the plurality of patients using a computing device including a processor and memory. At least a portion of the plurality of patients may be classified based on risk using one or more of the data trends. The patient may be selected for treatment from a health care professional based on the patient classification.

In some variations, an intervention plan may be generated for the selected patient using one or more of the data trends, and the intervention plan may be output to the health care professional. In some variations, a set of one or more prompts may be generated to modify the device settings of a patient computing device based on the risk. In some of these variations, the method may include the steps of generating one or more patient trends by analyzing one or more of the data trends among the plurality of patients. In some of these variations, the method may include the steps of generating one or more prompt trends by analyzing one or more of the patient trends against the set of prompts, classifying the prompts based on prompt effectiveness, wherein effectiveness is based on one or more of the prompt trends, and outputting a selected prompt from the offset of prompts to the patient computing device based on the effectiveness of the selected prompt. In some variations, a support group may be generated for the selected patient based on the patient classification. The support group may comprise at least one other patient from the plurality of patients. In some of these variations, a prompt may be generated for the selected patient to join and/or communicate with the support group.

Also described here are devices. In some variations, a device is provided, and may include a transceiver configured to receive analyte data generated by an analyte measurement device and patient data generated by a patient measurement device. A controller may be coupled to the transceiver. The controller may include a processor and a memory. The controller may be configured to generate one or more data trends by analyzing the analyte data against the patient data, generate a prompt to modify one or more patient computing device settings in response to one or more of the data trends, and output the prompt to a patient computing device using the transceiver. The devices and systems described herein may execute one or more steps of the methods described in more detail herein.

### BRIEF DESCRIPTION OF THE DRAWINGS

FIGS. 1A-1B are illustrative front views and perspective bottom views, respectively of a variation of an analyte measurement device. FIG. 1C is an illustrative perspective view of a cartridge that may be used with an analyte measurement device described here.

FIG. 2 is a block diagram of a variation of a disease management system.

FIGS. 3A-3C are illustrative flow charts of variations of a disease management process.

FIG. 4 is a set of illustrative variations of a graphical user interface.

FIGS. 5A-5E is another set of illustrative variations of a graphical user interface.

FIGS. 6A-6C is another set of illustrative variations of a graphical user interface.

FIGS. 7A-7B is another set of illustrative variations of a graphical user interface.

FIGS. 8A-8B is another set of illustrative variations of a graphical user interface.

FIG. 9 is another illustrative variation of a graphical user interface.

### DETAILED DESCRIPTION

Described here are systems, devices, and methods for managing a chronic disease using data analysis from a plurality of measurement devices. The data analysis may be used to generate an actionable prompt that may suggest a tangible step that the patient take such as making a change in behavior and/or a change in the setting of a device that the patient uses to manage their condition. The data gathered by the measurement devices may be output to the patient along with context to give meaning to the data as well as an action to be performed by the patient and/or system appropriate to the patient's condition. The data and/or action to be performed may be generated in view of analysis of the patient's data by itself or in combination with patient data from a set of patients.

Generally, the systems described here comprise an analyte measurement device, patient measurement device, and one or more of a computing device, remote server, and database. The measurement devices may generate data that is transmitted to the computing device, remote server, and/or database for processing and analysis. Data analysis may include trend analysis to find relationships and patterns between the data sets generated by the measurement devices as well as between patients. Trend analysis of different data sets (e.g., glucose, activity, drug, nutrition, health, etc.) may provide holistic insight to a patient and/or their care team (e.g., health care professional, coach, support group, family) of the patient's health over time. In some cases, the patient's analyzed data and trends may be used to aid one or more of the patient, health care professional, and other similar patients on actions that may be taken to improve health outcomes. The results of the analysis may be used to generate one or more prompts to output to the patient and/or health care professional. For example, data analysis showing that a patient is exhibiting a potentially dangerous trend may be used to output a patient prompt advising them to schedule an appointment with a health care professional, and/or to add an activity notification through their computing device to encourage more physical activity. Additionally or alternatively (e.g., concurrently), a prompt may be output to the patient's health care professional, family members, and/or other support group notifying them of the patient's condition and optionally suggesting appropriate intervention steps that they may take. As another example, data analysis showing that a patient is on a positive trend may be used to generate a prompt providing positive reinforcement to the patient and/or a reduction in the frequency of analyte testing.

In some variations, the patient and/or health care professional may receive a prompt from the system to take action to modify the settings of one of the devices in response to the trend analysis. The data analysis and prompts may be generated using device data corresponding to one patient or a plurality of patients. In some variations, a health care professional may use the data analysis of a plurality of patients to classify the patient to one or more subsets. The subsets may be used to prioritize attention and resources, thereby potentially increasing efficiency and lowering costs. For example, a set of high risk patients may be classified as high priority and given personal attention (e.g., from a group of health care professionals and/or coaches) while low risk patients may receive an automated message (e.g., managed by one health care professional and/or coach). In some variations, the effectiveness of each of a set of prompts provided to a set of patients may be analyzed (e.g., where prompt effectiveness may be correlated with increased compliance and/or suggested behavior modification) such that the set of prompts may be classified based at least in part on effectiveness. More effective prompt strategies may thereafter be given higher priority.

As used herein, a specific output and any data or signal corresponding thereto will be referred to as a “prompt.” A specific user input and any data or signal corresponding thereto will be referred to as a “command.” In some variations, the prompt may suggest a command for the user. For example, a prompt may output a command (e.g., actionable suggestion, recommendation, observation) that a user may affirmatively input to a device. For example, a prompt may be displayed on a touch screen device of the patient and suggest that the patient discuss their condition with a health care professional along with display of a suggested command to schedule an appointment with their health care professional (e.g., “Do you wish to schedule a doctor's appointment?”). The user may confirm execution of the command to schedule an appointment by selecting a corresponding icon on the touch screen. In some variations, the prompt may inform the user of a command executed without user input/confirmation, such as commands executed in an emergency situation.

### I. Systems

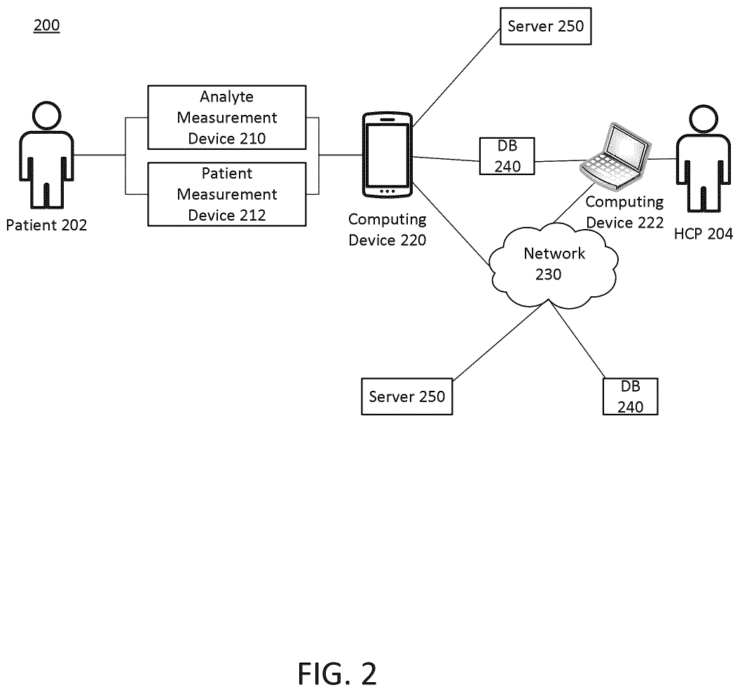

A disease management system may include one or more of the components necessary to measure and analyze patient data using the devices as described herein. In addition, the analysis may be used to generate and output a prompt to encourage healthy behavior and compliance. FIG. 2 is a block diagram of a variation of a disease management system (200). The system (200) may comprise an analyte measurement device (210) and a patient measurement device (212) configured to measure patient (202) characteristics such as blood glucose and physical activity (e.g., steps taken, heart rate). The analyte measurement device (210) and patient measurement device (212) may be coupled to a computing device (220) through one or more wired or wireless communication channels. The computing device (220) may be coupled one or more networks (230), databases (240), and/or servers (250). The network (230) may comprise one or more databases (240) and servers (250). In some variations, a health care professional (HCP) (204) may be coupled one or more networks (230), databases (240), and servers (250) through a respective computing device (222). In some variations, the measurement devices (210, 212) may be coupled directly to any of the network (230), database (240), server (250), or each other. Processing and analysis may be performed at any one of the devices of the system (200) or distributed throughout a plurality of devices.

### Analyte Measurement Device

Generally, the analyte measurement devices described here are configured to perform analyte measurement operation in which the concentration of one or more analytes in a fluid sample is measured. For example, an analyte measurement device may be configured to collect a fluid sample from a sampling site, transport the fluid sample to an analysis site, and analyze the fluid sample. Analysis of a fluid sample may include determining the concentration of one or more analytes in the sample, such as one or more hormones, proteins, enzymes, toxins, drugs, other molecules, or the like. In some variations, the analyte measurement devices described here may be configured to measure the glucose concentration of one or more blood samples or other glucose-containing solutions. Analyte data including fluid sample analysis may be transmitted to one or more computing devices as described herein. The analyte measurement device may be controlled from one or more computing devices.

When the analyte measurement device is configured to collect a fluid sample from a sampling site, the device may be configured to collect a fluid sample from any suitable sampling site. Examples of suitable sampling sites include, but are not limited to, one or more body sites (e.g., fingers, toes, other skin surfaces, or the like) or one or more artificial containers (e.g., a vial holding a control solution or a body fluid sample). The fluid sample may comprise any suitable fluid, such as, for example, one or more solutions (e.g., a control solution), mixtures, body fluids (e.g., blood, saliva, or the like), combinations thereof, and the like.

In some variations, an analyte measurement device as described here may be fully integrated, in that the device may contain all of the components necessary for collecting, transporting, and analyzing a fluid sample. For example, the systems described here may comprise one or more of the devices described in U.S. patent application Ser. No. 14/311,114, filed Jun. 20, 2014 and titled “ANALYTE MONITORING SYSTEM WITH AUDIBLE FEEDBACK,” U.S. patent application Ser. No. 13/566,886, filed Aug. 3, 2012 and titled “DEVICES AND METHODS FOR BODY FLUID SAMPLING AND ANALYSIS,” U.S. Pat. No. 7,004,928, filed Apr. 23, 2002 and titled “AUTONOMOUS, AMBULATORY ANALYTE MONITOR OR DRUG DELIVERY DEVICE,” and U.S. Pat. No. 8,012,103 and titled “CATALYSTS FOR BODY FLUID SAMPLE EXTRACTION,” the contents of each of which are hereby incorporated by reference in their entirety. It should also be appreciated that the analyte measurement devices described here may be configured to perform only a subset of the collecting, transporting, and analyzing operations associated with analysis of a fluid sample.

For example, the analyte measurement device may comprise a fully integrated wireless meter. The meter may comprise a meter housing and one or more strip-free cartridges, which will be described in more detail herein. In some variations, the meter may be configured to collect and analyze a plurality of fluid samples. For example, in some variations, a cartridge may comprise one or more cells, some or all of which may contain one or more sampling arrangements for collecting a fluid sample, as described in more detail below. The meter may be further configured to audibly, visually, and/or otherwise provide one or more results from the sample analysis.

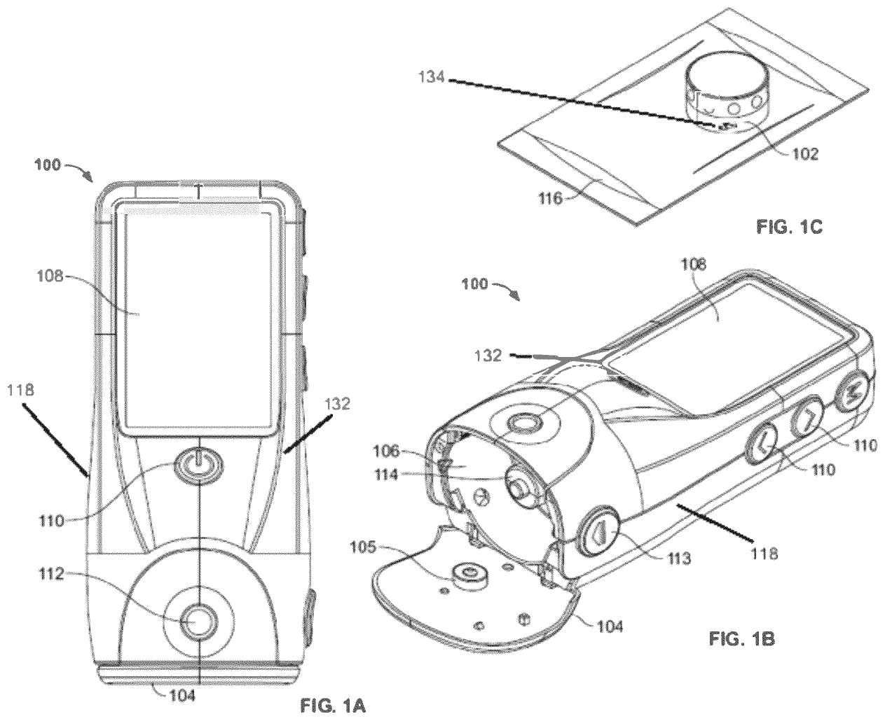

FIGS. 1A-IC show an illustrative variation of an exemplary integrated meter (100). Specifically, FIGS. 1A and 1B show a front view and a bottom perspective view, respectively, of a meter housing (118), while FIG. 1C shows a perspective view of the cartridge (102). While shown in FIG. 1C as being stored in a sealed or scalable pouch (116), the cartridge (102) may be stored in any suitable container, and may be removed prior to use. As shown in FIGS. 1A and 1B, the meter housing (118) may comprise a door (104) with a cartridge-engagement projection (105), a cartridge-receiving chamber (106) or cavity, a cartridge ejection button (113), a display (108), buttons (110), a port (112), and a tower (114). The meter need not include each of these features, and it should be appreciated that the meter may comprise any combination of these features. The meter (100) may further comprise one or more imaging systems (not shown), and internal mechanisms or components (e.g., memory, circuitry, actuators, batteries, vacuum pumps, sensors, combinations thereof, etc.) for operating the meter and/or facilitating a testing procedure

A cover or door (104) may be opened to reveal a cartridge-receiving chamber (106), as shown in FIG. 1B. A cartridge (102) may be placed inside of cartridge-receiving chamber (106), and the door (104) may be closed to temporarily enclose the cartridge (102) within the meter housing (118). When placed inside of the meter housing (118), one or more portions of the cartridge (102) may engage one or more components of the meter housing (118). In some variations, the meter housing (118) may comprise one or more features that may facilitate self-alignment of the cartridge (102) as it is placed in the cartridge-receiving chamber (106). For example, in some variations the cartridge (102) may comprise a recess (not shown). When the cartridge (102) is placed inside of the cartridge-receiving chamber (106), a portion of the tower (114) may fit within or otherwise engage the recess of the cartridge (102). This engagement may help to hold the cartridge (102) in place relative to meter housing (118). Conversely, in some variations, the cartridge (102) may comprise one or more projections (not shown) that may engage one or more recesses (not shown) in the cartridge-receiving chamber (106) or other portion of the meter housing (118). Additionally or alternatively, one or more magnets may hold the cartridge (102) in place relative to the meter housing. A cartridge (102) need not be placed inside of a meter housing (118) (e.g., via a cartridge-receiving chamber) to engage the meter housing (118). For example, in some variations, a cartridge (102) may attach to or otherwise engage one or more external surfaces of a meter housing (118).

Any suitable cartridge may be used with the meters. For example, in some variations, the meter may comprise one or more of the cartridges described in U.S. patent application Ser. No. 14/311,114, filed Jun. 20, 2014 and titled “ANALYTE MONITORING SYSTEM WITH AUDIBLE FEEDBACK,” U.S. patent application Ser. No. 11/529,614, titled “MULTI-SITE BODY FLUID SAMPLING AND ANALYSIS CARTRIDGE,” and U.S. Pat. No. 8,231,832, titled “ANALYTE CONCENTRATION DETECTION DEVICES AND METHODS,” the contents of each of which is hereby incorporated by reference in its entirety

The meter housing (118) may be configured to house a speaker and/or a microphone (not shown) and a controller (not shown), although it should be appreciated that the speaker, microphone, and/or controller may in some instances be partially housed in the housing (118), may be externally attached to the housing (118), or may be part of a separate device (i.e., headphones, smartphone, computer, tablet, etc.) that communicates with the meter (100) either wirelessly or through a wired connection. As depicted in FIG. 1A, the meter housing (118) may additionally comprise a display (108) (e.g., for visually providing information to a patient), buttons (110) (e.g., for powering on/off the device, inputting information into the device, etc.) and a port (112) (e.g., through which a fluid sample may be collected), such as those described in U.S. patent application Ser. No. 13/566,886, which was previously incorporated by reference in its entirety. Additionally or alternatively, the meter (100) may in some instances further comprise one or more imaging systems (not shown), and internal mechanisms or components (e.g., memory, circuitry, actuators, batteries, vacuum pumps, sensors, combinations thereof, etc.) for operating the meter (100) and/or facilitating testing of a fluid sample The analyte measurement devices described here need not include each of these features, and variations of these devices may comprise any combination of these features. Additionally, the analyte measurement device may be configured to receive general information useful in determining when testing occurs (e.g., time of day, date, location, etc.) as is described in more detail in U.S. patent application Ser. No. 12/457,332, titled “MEDICAL DIAGNOSTIC DEVICES AND METHODS,” the content of which is hereby incorporated by reference in its entirety.

### Patient Measurement Device

A patient measurement device as used herein may refer to any device configured to measure and/or analyze one or more characteristics of a patient. A patient measurement device may, for example, measure a patient analyte, activity, and nutrition data. Non-limiting examples of patient measurement devices include a wearable device (e.g., pedometer or other activity tracker), sleep tracker, hydration tracker, blood pressure monitor, heart rate monitor, cholesterol monitor, A1c test device, scale, refrigerator, geolocation devices (e.g., GPS, GLONASS), smartphone, smart refrigerator, PC, implantable device, ingestible device, and other diagnostic devices. Patient data generated by the patient measuring device may include, but is not limited to, nutrition data (e.g., meal marking, consumed carbohydrate count, consumed calorie count, etc.), activity or exercise (e.g., calories burned, steps performed, degree or intensity of activity (e.g., based on heart rate levels), duration of activity, etc.), duration or quality of sleep, patient weight, oral or other medications, insulin (e.g., pen, syringe, pump, inhalable, etc.), mood/feeling, stress, hydration, and the like. The data generated by the patient measurement device may be transmitted to any of the devices of the system (200), and may include one or more of the features, elements, and/or functionality of the computing devices, as described herein. For example, the patient measurement device may comprise a controller comprising a processor and memory to perform data analysis on the patient data generated by the patient measurement device and a communication interface configured to transmit the measurement data to another device. The patient measurement device may couple to a device using any known wired or wireless connection method and communication protocol.

### Computing Devices

Generally, the computing devices described here may comprise a controller comprising a processor (e.g., CPU) and memory (which can include one or more computer-readable storage mediums). The processor may incorporate data received from memory and user input to control one or more components of the system (e.g., analyte measurement device (210), patient measurement device (220)). The memory may further store instructions to cause the processor to execute modules, processes and/or functions associated with the methods described herein. As used herein, a computing device may refer to any of the computing devices (220, 222), databases (240), and servers (250) as depicted in FIG. 2. In some variations, the memory and processor may be implemented on a single chip. In other variations, they can be implemented on separate chips.

A controller may be configured to receive and process the measurement data from the analyte measurement device and patient measurement device. The computing devices may be configured to receive, compile, store, and access data. In some variations, the computing device may be configured to access and/or receive data from different sources. The computing device may be configured to receive data directly input by a patient and/or it may be configured to receive data from separate devices (e.g., a smartphone, tablet, computer) and/or from a storage medium (e.g., flash drive, memory card). The computing device may receive the data through a network connection, as discussed in more detail herein, or through a physical connection with the device or storage medium (e.g. through Universal Serial Bus (USB) or any other type of port). The computing device may include any of a variety of devices, such as a cellular telephone (e.g., smartphone), tablet computer, laptop computer, desktop computer, portable media player, wearable digital device (e.g., digital glasses, wristband, wristwatch, brooch, armbands, virtual reality/augmented reality headset), television, set top box (e.g., cable box, video player, video streaming device), gaming system, or the like.

The computing device may be configured to receive various types of data. For example, the computing device may be configured to receive a patient's personal data (e.g., gender, weight, birthday, age, height, diagnosis date, anniversary date using the device, etc.), a patient's testing history (e.g., number of tests completed, time each test was completed, date each test was completed, pre or post prandial test markings, how many tests a patient has completed consecutively, etc.), a patient's results history (e.g., glucose level at time test was taken), a patient's diet information (e.g., what a patient had to eat each day, number of alcoholic beverages, amount of carbohydrates consumed, etc.), a patient's exercise information (e.g., if a patient exercised, when the patient exercised, duration of exercise, what type of exercise the patient completed (e.g. biking, swimming, running, etc.), exertion level of the exercise (e.g., low, medium, high), a patient's heart rate during exercise, etc.), general health information of other similarly situated patients (e.g., typical test results for a similar patient at a similar time of day, average of test results for a similar patient after exercise, etc.), or any other information that may be relevant to a patient's treatment. In some variations, the computing device may be configured to create, receive, and/or store patient profiles. A patient profile may contain any of the patient specific information previously described.

While the above mentioned information may be received by the computing device, in some variations, the computing device may be configured to calculate any of the above data from information it has received using software stored on the device itself, or externally. In some variations, the computing device may be configured to identify patterns in patient behavior, use the identified patterns to predict future patient behavior, and provide prompts to the patient relating to the identified patterns, as is described in more detail in U.S. patent application Ser. No. 12/457,332, titled “MEDICAL DIAGNOSTIC DEVICES AND METHODS,” which was previously incorporated by reference in its entirety. In some instances, the computing device may use the patterns and/or data analysis to help warn about, or prevent the occurrence of one or more glucose events. A glucose event may occur any time a patient's glucose is above or below an expected level or is outside a specified range. In some variations, a glucose event may be a hypoglycemic event or a hyperglycemic event. In some variations, the computing device may be configured to compare the patient's personal data, testing history, diet information, exercise information, or any other relevant information, to a patient's historical data (e.g. prior test data, patient's historical trends, etc.), data preloaded onto the computing device that has been compiled from external sources (e.g. medical studies), or data received from a set of separate devices (e.g., historical data or data compiled from external sources). In some instances, the warning or notification may include instructions to perform a test, seek medical attention, and/or to cat or drink something.

The processor may be any suitable processing device configured to run and/or execute a set of instructions or code and may include one or more data processors, image processors, graphics processing units, physics processing units, digital signal processors, and/or central processing units. The processor may be, for example, a general purpose processor, Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions associated with the communication device, such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the computing device (220, 222) may further comprise a communication interface configured to permit a patient and/or health care professional to control one or more of the devices of the system. The communication interface may comprise a network interface configured to connect the computing device to another system (e.g., Internet, remote server, database) by wired or wireless connection. In some variations, the communication device (220, 222) may be in communication with other devices via one or more wired and/or wireless networks. In some variations, the network interface may comprise a radiofrequency receiver, transmitter, and/or optical (e.g., infrared) receiver and transmitter configured to communicate with one or more devices and/or networks. The network interface may communicate by wires and/or wirelessly with one or more of the measurement devices (210, 212), network (230), database (240), and server (250).

The network interface may comprise RF circuitry may receive and send RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the computing and measurement devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

The communication interface may further comprise a user interface configured to permit a user (e.g., patient, predetermined contact such as a partner, family member, health care professional, coach, etc.) to control the computing device. The communication interface may permit a user to interact with and/or control a computing device directly and/or remotely. For example, a user interface of the computing device may include an input device for a user to input commands and an output device for a user to receive output (e.g., patient data analysis and prompts on a display device).

An output device of the user interface may output data analysis and actionable prompts corresponding to the patient and may comprise one or more of a display device and audio device. For example, a video conference between the patient and a health care professional may be facilitated using the display device of the computing device. A display device may permit a user to view trend analysis and/or other data processed by the controller. Data analysis generated by a server (250) may be displayed by the output device (e.g., display) of the computing device (220, 222). Measurement data from one or more measurement devices (210, 212) may be received through the network interface and output visually and/or audibly through one or more output devices of the computing device (220). In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output patient data, system data, alarms and/or notifications. For example, the audio device may output an audible alarm when measured data (e.g., blood glucose) falls outside a predetermined range or when a malfunction in the analyte measurement device (210) is detected. In some variations, an audio device may comprise at least one of a speaker, piezoelectric audio device, magnetostrictive speaker, and/or digital speaker. In some variations, a user may communicate with other users using the audio device and a communication channel. For example, a patient may form an audio communication channel (e.g., VOIP call) with a remote health care professional.

In some variations, the user interface may comprise an input device (e.g., touch screen) and output device (e.g., display device) and be configured to receive input data from one or more of the measurement devices (210, 212), network (230), database (240), and server (250). For example, user control of an input device (e.g., keyboard, buttons, touch screen) may be received by the user interface and may then be processed by processor and memory for the user interface to output a control signal to one or more measurement devices (210, 212). Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the computing device.

### Network

In some variations, the systems and methods described herein may be in communication with other computing devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication.

### II. Methods

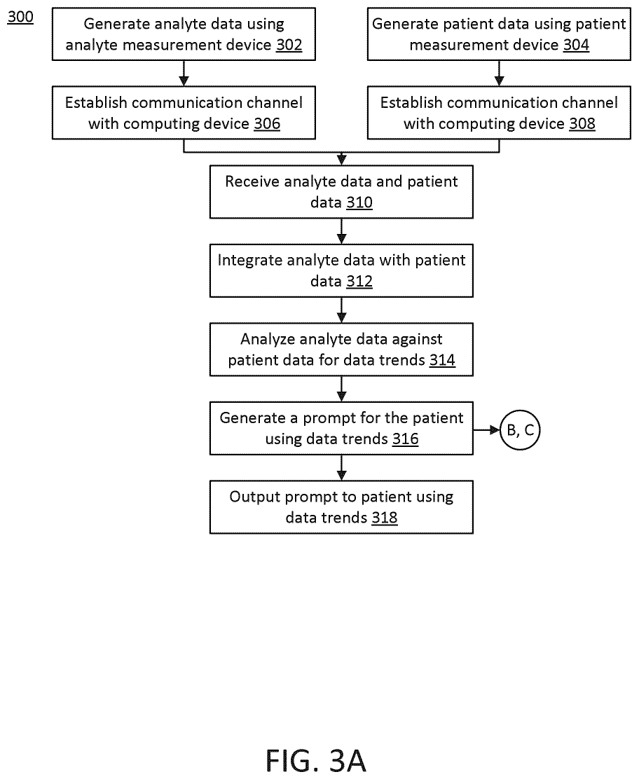

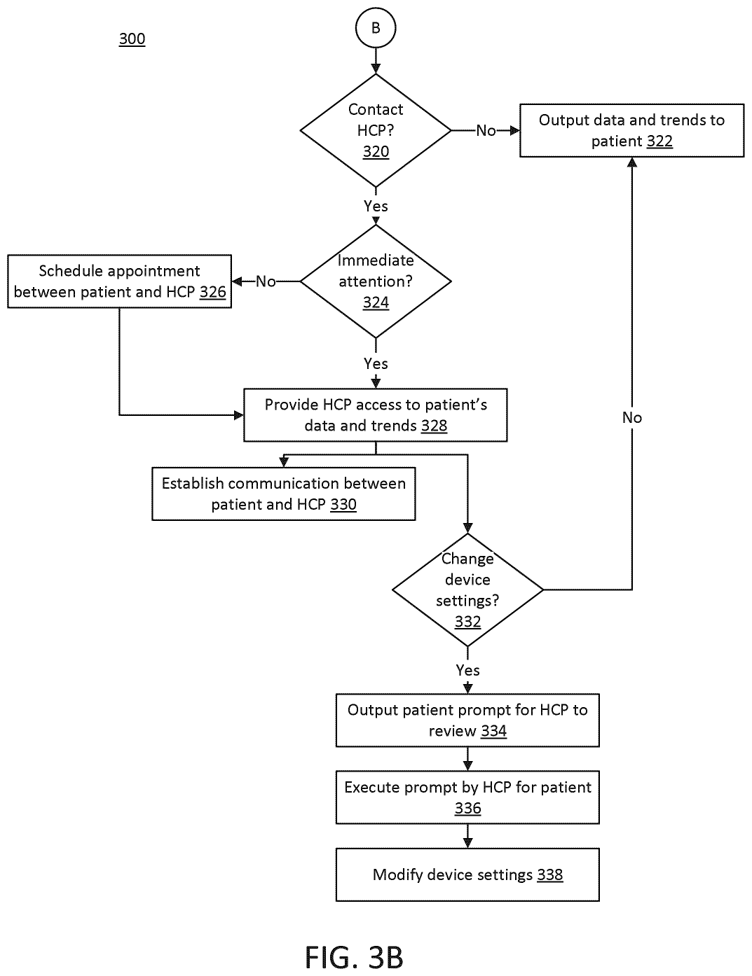

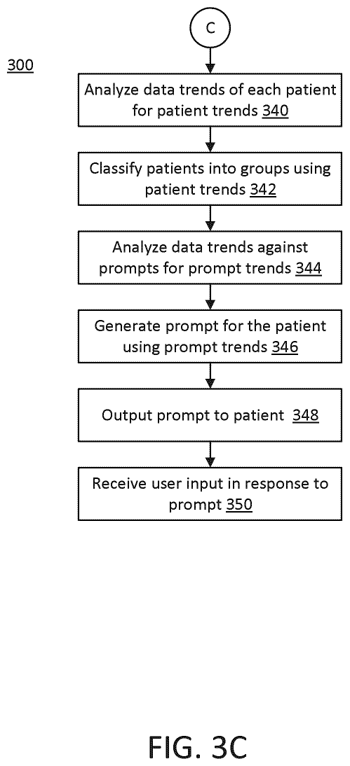

Also described here are methods for monitoring a chronic condition of a patient using the systems and devices described herein. Generally, the methods described here include receiving data from an analyte measurement device and a patient measurement device, generating a data trend by analyzing the data, and modifying device settings in response to the data trend. It should be appreciated that any of systems and devices described herein may be used in the methods described herein. FIGS. 3A-3C are flowcharts that generally describe a patient analysis and monitoring process (300).

### Managing a Chronic Condition of a Patient

The process (300) may include generating analyte data using an analyte measurement device of a patient (302) and generating patient data using a patient measurement device of a patient (304). For example, patient measurement devices may include one or more of a wearable device (e.g., activity tracker), sleep tracker, hydration tracker, blood pressure monitor, heart rate monitor, cholesterol monitor, A1c test device, scale, geolocation devices (e.g., GPS, GLONASS), smartphone, tablet, smart refrigerator, implantable device, ingestible device, and other diagnostic devices. Furthermore, patient data may be generated on a computing device running an application such as a nutrition (e.g., calorie, meal) analysis application that may, for example, be manually input to the nutrition application. Therefore, in some cases, the patient measuring device need not necessarily generate patient data using sensors. As used herein, a patient measuring device may also refer to devices storing patient data accessible through a network (e.g., website, remote server, cloud database, etc.). For example, patient data may be pulled from a patient's activity tracker platform.

A communication channel may be established between the analyte measurement device and a computing device (306). A communication channel may be established between the patient measurement device and a computing device (308). In some variations, a plurality of measurement devices corresponding to a plurality of patients may generate data and establish respective communication channels to a plurality of respective computing devices. The communication channel may be a wired or wireless connection and use any communication protocol including but not limited to those described herein. The communication channel may be established at predetermined intervals based on one or more of time (e.g., hourly, daily, weekly, etc.), device usage (e.g., after every test measurement taken, upon device power on, before entering sleep mode, memory usage, battery level, establishment of a communication channel, etc.), measurement data analysis (e.g., falling outside a predetermined range), request for connection, and the like.

Additionally or alternatively, a communication channel may be manually established by a user (e.g., patient, family member, health care professional, coach, etc.) at any desired time. The measurement devices may establish the communication channel directly or indirectly with one or more computing devices (e.g., smartphone, database, remote server, Internet, and the like) as described herein. For indirect connections, the intermediary device(s) may establish additional communication channels. For example, a glucose monitoring device may establish a connection to a smartphone to initially transfer glucose data. The smartphone may then transfer the glucose data to a cloud database and/or any other computing device (e.g., remote server). In some variations, the analyte monitoring device may attempt to find a computing device to establish a communication channel for a predetermined amount of time (e.g., one minute) after completion of an analyte test. The analyte monitoring device may preferably connect to a recognized and/or authorized computing device such as a patient's smartphone and/or desktop PC.

Analyte data and patient data may be received at the computing device (310). For example, data may be transmitted wirelessly using a Bluetooth protocol and by wire through a USB connection. In some variations, once a communication channel has been established between the measurement device and the computing device, analyte data and patient data may be automatically transmitted without patient input and/or notification. For example, the analyte measuring device may transmit all the generated data or a subset of data (e.g., set of data not yet received by the computing device) in the background while the patient uses other applications on their smartphone. Additionally or alternatively, the computing device may receive the last test result, all test results, test results from a predetermined time frame, and the like. The received data may be further transmitted to a cloud database. Analyte and patient data stored on a cloud database may be accessible from any account and/or device that is granted access to that data. In some variations, a patient's computing device may connect to another service/platform containing patient data (e.g., activity tracker data, meal data) to receive that data.

Analyte data may be integrated with patient data (312). Data integration may comprise formatting the data to permit comparison and analysis across sets of data generated from different devices. In some variations, data integration may be performed by, for example, a controller of a remote server (220) and/or computing device (222) separate from the analyte measurement device (210), patient measurement device (212), and computing device (220). In some variations, analyte data and patient data may be integrated to permit comparison on the same time scale. For example, analyte data and patient data may undergo time stamp synchronization to standardize the time format of the data sets and permit, for example, activity data to be linked with glucose measurements. In some variations, glucose may be measured in mg/dL and may range from about 30 mg/dL to about 500 mg/dL and above. Activity may be measured along a time axis. Exertion may be estimated from heart rate zone data. Carbohydrates may be measured in grams. Weight may be measured in pounds or kilograms.

Additionally or alternatively, data integration may comprise range normalization. For example, each data point may be scaled to a predetermined range (e.g., 0-100) to permit an algorithm to manipulate the data irrespective of a unit of measure. Furthermore, the data sets may be integrated to permit comparison of data of one patient against the data of other patients. Integration of the data may be performed across one or more devices. Data integration may further comprise resolving data conflicts (e.g., manually generated data vs. device generated data, corrupt data, missing data, and the like). In some variations, data integration may be performed for data sets corresponding to a plurality of patients.

Data trends may be generated by analyzing analyte data against patient data for each patient (314). Data analysis of data from different measurement devices may identify useful trends between analyte data and patient data for output to users (e.g., patients and health care professionals) and permit generation of actionable prompts. For example, activity data from a fitness tracker (e.g., steps taken, heart rate, and the like) may be analyzed against blood glucose measurements from a blood glucose monitor and used to generate a data trend indicating that the patient's blood sugar level is inversely related to their activity. For example, a trend may be generated showing that during more sedentary periods of time, a patient's blood sugar is higher. Performing data analysis on or more computing devices (e.g., a remote server) may increase the speed of analysis. In variations where analysis is performed on a remote server, the analysis and trend generation steps may be updated just on the server without requiring an update to the software of the computing device (e.g., smartphone) of each patient. In some variations, one or more of the data, analysis, and trends described herein may be graphically output (e.g., charts, graphs, symbols, animations) to a user (e.g., patient, predetermined contact, health care professional, coach) on any device such as the computing devices as described in more detail herein.

Similar to data integration, data and trend analysis as described herein may be performed by, for example, a controller of a remote server (220) and/or computing device (222) separate from the analyte measurement device (210), patient measurement device (212), and computing device (220).

In some variations, data and trend analysis may be based on regression analysis techniques (e.g., linear regression, curve fitting). Trend analysis may include, for example, analysis of analyte data parameters including testing time (e.g., time since last test), test frequency (e.g., three per day), and blood glucose values against activity data and/or meal data. In these examples, trend analysis may indicate that high blood sugar correlates with inactivity and/or poor meal choices (e.g., alcohol consumption). Longer term trends may be generated as well, such as seasonal trends indicating higher glucose levels around the holidays, inclement weather, and the like.

In some variations, a wellness indicator (e.g., wellness value) for a patient may be generated to provide a simplified metric to help users more easily or quickly gauge the patient's overall health or specific area of health, and/or progress in controlling a health condition such as diabetes. The wellness indicator may be a simplified indicator of general overall health that considers and aggregates a set of health characteristics, where each health characteristic may generally be viewed as having a positive or a negative impact on the wellness indicator. The wellness indicator may be scaled to a predetermined range. For example, the wellness indicator may be a number (e.g., between 0 and 5, between 0 and 10, between 0 and 100, percentile ranking), a letter grade (e.g., A, B, C, D, F, etc.), a color (e.g., red, orange, yellow, green), descriptor (e.g., excellent, good, ok, fair, poor, improving, maintaining, decreasing, etc.), graphics (e.g., smiley face, neutral face, unhappy face, animation, etc.) sound effect or musical sequence (e.g., beep, tone, rising scale, falling scale, musical trill, applause, etc.), vibrations (e.g., number of discrete vibrations, duration of vibration, etc.), combinations thereof, and the like.

The wellness indicator may be calculated in any suitable manner. In some variations, a wellness indicator may be based on a set of analyzed patient trends (e.g., blood glucose trend, blood glucose and activity trend, stress and nutrition trend, etc.), where each patient trend is weighted with a respective weighting factor. For example, the wellness indicator may be calculated by weighting one or more of glucose data, testing compliance data, a number of hypoglycemic events, nutrition data, activity data, weight data, sleep data, hydration data, combinations thereof, and the like, over a predetermined period of time (e.g., preceding 7 days, 15 days, 30 days, seasons, etc.). Although in some variations the weighting factors may be similar for all patients, in some variations the weighting factors may be tailored for a particular patient or patient subpopulation based on a patient characteristic. For example, an overweight patient may have a higher weighting factor for a patient weight trend compared to a lighter patient, which may reflect a greater importance of patient weight for the overweight patient's wellness indicator. The weighting factors may remain constant or may be adjusted (e.g., manually such as by a health care professional).

In some variations, the wellness indicator may comprise one or more weighted parameters or characteristics (e.g., weighted by scale factors) and function as an indicator of diabetes control (e.g., diabetes wellness indicator). Parameters contributing to the wellness indicator may include, for example, glucose metrics (e.g., glucose measurements, glucose trends, etc.), glucose events (e.g., number of hypoglycemic and/or hyperglycemic events), activity data, nutrition data (e.g., carbohydrates consumers, hydration, etc.), sleep data (e.g., average amount of sleep per day), and doctor visits, etc. Some parameters may be scaled differently than others. For example, greater weight may be given to one or more primary characteristics directly related to blood glucose health while secondary factors such as sleep, activity, doctor visits, etc. may be incorporated within the overall wellness indicator.

In some variations, the wellness indicator may be output on a patient computing device and/or other device (e.g., to a patient, a user that is part of the patient's set of predetermined contacts as described in further detail below, a health care professional, coach, etc.). For example, the wellness indicator may be displayed on a display of a computing device, communicated through a speaker or other audio device, communicated through tactile sensations (e.g., vibration).

In some variations, a graph of the wellness indicator over time may be output on a patient computing device. In some variations, the wellness indicator may optionally include a prompt providing an actionable suggestion of how the patient may increase their wellness indicator and/or observations on how the wellness indicator is derived. For example, the prompt may suggest the patient schedule an appointment with their health care professional within the next month, add a 30 minute walk after lunch, or add an additional testing reminder based on a wellness indicator within a predetermined range (e.g., fair and decreasing). The prompt may include observations that may provide additional context to the wellness indicator. For example, the observation may indicate that an improvement in wellness indicator over the last three months is correlated to an increase in a number of activity minutes per week. In one specific implementation, a wellness indicator may be calculated according to exemplary Equation (1) below:

$$Ws = 100 - a ( s \cdot d ( glucose over 30 days ) ) - b ( \vert target glucose - avg ( glucose over 30 days ) \vert ) - c ( number of hypoglycemic readings over 30 days ) - d ( number of hyperglycemic readings over 30 days ) + e ( \% of readings in target range - 60 \% ) + f ( number of glucose measurements over 30 days ) + g ( \frac{minutes of activity over previous 7 days}{60} ) - h ( minutes of activity - target minutes of activity ) - i ( grams of carbohydrates consumed over previous day ) - j ( \begin{matrix}grams of carbohydrates consumed over previous day \\ above target grams of carbohydrates consumed over previous day\end{matrix} ) - k ( BMI ) + l ( number of meals marked ) + m ( \frac{number of hours of sleep over 7 days}{7} ) + n ( number of doctor visits over previous 365 days ) + p ( number of eye exams over previous 365 days ) + q ( number of diabetic foot exams over previous 365 days ) , \qquad ( 1 )$$

where a, b, c, d, e, f, g, h, i, j, k, l, m, n, p, and q are scale factors, s.d. is standard deviation, and BMI is Body Mass Index.

For example, the scale factors may be configured such that a≤b≤c≤d≤e≤f≤g≤h≤i≤j≤k≤l≤m≤n≤p≤q. For a new patient having less than 90 days of testing history and/or patient data, the scale factors g, h, i, j, k, l, m, n, p, and q may be set to zero or a nominal value in order to emphasize blood glucose measurements in the wellness indicator. For other patients with a history of low minutes of activity, scale factors g and h may be substantially equal to one of the glucose scale factors, a, b, c, d, e, and f in order to emphasize physical activity in the wellness indicator. Scale factors may be hard-coded into software, received via user input (e.g., through a web portal or other user interface on a user computing device), or input into the wellness indicator determination in any suitable manner.

In some variations, the scale factors may be configured based on one or more of type of diabetes, age, and time diagnosed with diabetes. In other variations, scale factors for a subgroup of patients may be configured to appropriately represent health or range of health for that subgroup. For example, scale factors for a subgroup of patients may be based at least partially on trend analysis of the subgroup. In some variations, other measures of dispersion/variability including, but not limited to, mean absolute deviation, interquartile range, and sample variance, may be used with and/or in place of standard deviation. It should be appreciated that the time intervals in Equation (1) are merely exemplary and may be shorter or longer (e.g., 7 days, 90 days), and similarly, the units of measurement (e.g., minutes, grams, etc.) used in Equation (1) may be suitably modified. For example, the time ranges for the grams of carbohydrates consumed and target grams of carbohydrates consumed may be any date range (e.g., 1 day, 7 days, 15 days, 30 days).

In some variations, analysis of analyte data and/or patient data may generate a data trend corresponding to an estimated of risk of a hypoglycemic event. A set of device settings and/or prompts may be output based on the estimated risk. For example, an estimated low risk condition for a hypoglycemic event over a predetermined length of time may correspond to a prompt to reduce analyte testing frequency while an estimated high risk condition for a hypoglycemic event may correspond to a prompt to immediately schedule an appointment with a health care professional. In some variations, analyte data and patient data may be analyzed to estimate a risk of a hypoglycemic event. Risk of a hypoglycemic event may be estimated based at least partially on patient activity, patient exertion level, and carbohydrates consumed. For example, a large difference between a patient's average blood glucose measurement and their current blood glucose measurement relative to their activity level and carbohydrate consumption may indicate a high risk for a hypoglycemic event. In one specific implementation, a numerical indication of risk of a hypoglycemic event may be estimated according to exemplary Equation (2) below:

$$\frac{( \frac{AvgGlu}{Current Glucose} ) * ( Act * Exe ) - ( Carbs * 4 )}{100} \qquad ( 2 )$$

where AvgGlu is the average glucose value over the last 90 days, Current Glucose is a current glucose value, Act is the number of minutes of patient activity, Exe is an exertion level ranging between 1 and 5 based on heart rate, and Carbs is the number of grams of carbohydrates consumed in the last 90 minutes. A higher value calculated per Equation (2) may, for example, indicate a higher risk of a hypoglycemic event. Although exemplary specific numbers are provided in connection with Equation 1, it should be understood that in other variations the equation may be modified (e.g., number of days, units for patient activity or exertion or nutrition, etc.).

In some variations, data analysis may include activity data and glucose data. For example, a patient having a low blood glucose measurement while also engaging in intense physical exercise may be at high risk for a hypoglycemic event. In particular, if a blood glucose measurement at a point in time is less than 150 mg/DL and Act*Exe>200, then the risk of hypoglycemia 10 hours post activity may increase. A patient meeting these thresholds may be classified as high-risk. It should be understood that other suitable threshold values may be implemented. If these thresholds are met, then a prompt may output a suggestion to set an additional glucose test at around the 10 hours post-glucose measurement, as described in more detail herein with respect to prompts. In some variations, the wellness indicator and estimated risk of hypoglycemia may be calculated by, for example, a controller of a remote server (220) and/or computing device (222) separate from the analyte measurement device (210), patient measurement device (212), and computing device (220).

A set of prompts may be generated for the patient based on the trends (e.g., data trends, patient trends) (316). In some variations, generation and transmission of a set of prompts may be performed by, for example, a controller of a remote server (220) and/or computing device (222) separate from the analyte measurement device (210), patient measurement device (212), and computing device (220). For example, a prompt may be configured and generated by a health care professional computing device (222) and transmitted to the patient computing device (220) when predetermined criteria (e.g., trend thresholds) are met. The generation and output of prompts as described herein may reduce the need for emergency hospital visits by identifying potentially dangerous situations and suggesting an action to prevent the situation from worsening without the time and expense of intervention by a health care professional. For example, the prompts described herein may reduce the need for a costly hospital visit by suggesting actions that the patient may take to improve their condition. Even multiple consultations with a health care professional as suggested through the prompts may be more cost effective than a single hospital visit. Some prompts may be informative (e.g., communicate observations or alerts), some prompts may be actionable (e.g., invite an action by the patient), and some prompts may be informative and actionable.

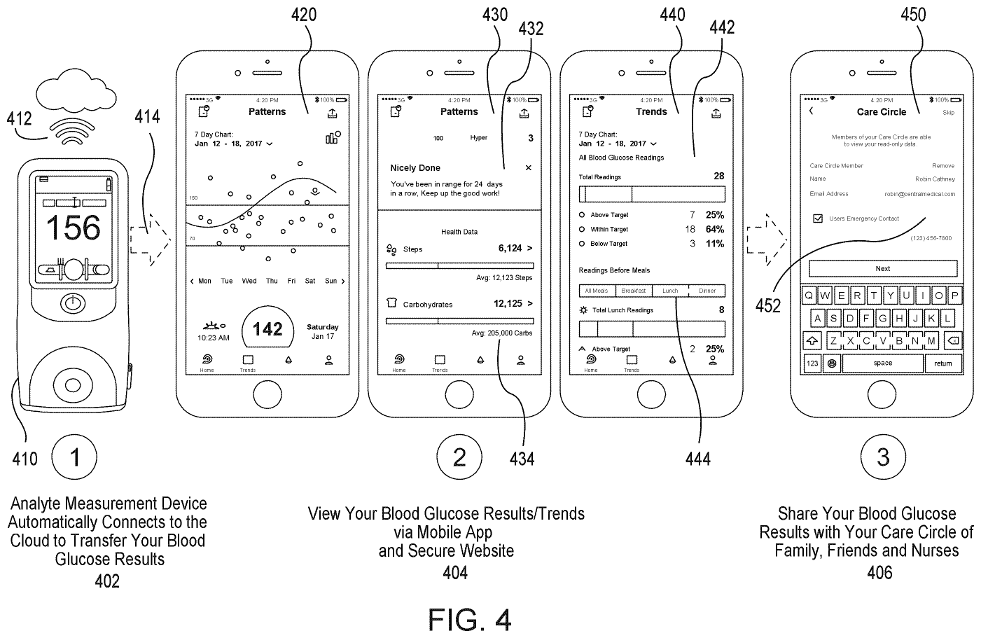

The prompt may, in some variations, function as an automated digital coach with suggestions and/or observations for improving health. The trend analysis may be presented to users along with an actionable suggestion to improve their health without any intervention from a human coach. In some variations, a prompt may indicate patient compliance with an expected testing regimen (or lack thereof), and further include encouragement to the patient to better comply with the regimen. For example, if a user is complying with the expected testing, diet, and/or exercise regimen (e.g., consecutively or consistently testing at prescribed times, step goal, intensity minutes goal, diet goals), the prompt may acknowledge and praise the patient using a GUI (see FIG. 4). In some variations, a prompt may ask the patient to share their accomplishments with one or more predetermined contacts (e.g., partner, family member, support group) and may optionally provide a prompt for them to respond. Conversely, if the user is not complying with the expected testing, diet, and/or exercise regimen, the prompt may inform the patient and/or one or more predetermined contacts of this. Furthermore, in some variations, greater deviations from target or expected goals may trigger prompts that are delivered more frequently to the patient and/or to more (or to certain kinds) of predetermined contacts.

The prompt may be output to the patient (318) on any accessible computing device such as an analyte measurement device and a computing device. The patient may use a graphical user interface (GUI) to view their data, analysis, and actionable suggestion (e.g., prompts) using one or more of a mobile application (e.g., iOS, Android), web browser accessing a secure website, and/or cloud computing solution. The patient may register an account through the application and login to access its functionality. The displayed prompt may provide information about patient testing history, results, and analysis. The analyte and patient data may be presented using the graphical user interface in one or more customizable formats that allow a patient to gain greater insight to their diabetes and overall health. For example, glucose trends may be plotted over time and may include target information, averages, and color coding. Glucose data may be displayed for a single day. Analyte data and patient data may be displayed in a table format. Target analysis may be presented in a pie chart and illustrate how well the patient is meeting their target goals. Information including one or more of health records, lab results, meal data, medication information, patient profile, insurance, and testing schedule may all be accessible through the GUI and used in data analysis. Additionally or alternatively, data, analysis, and/or prompts (e.g., notifications) may include texts and e-mails.

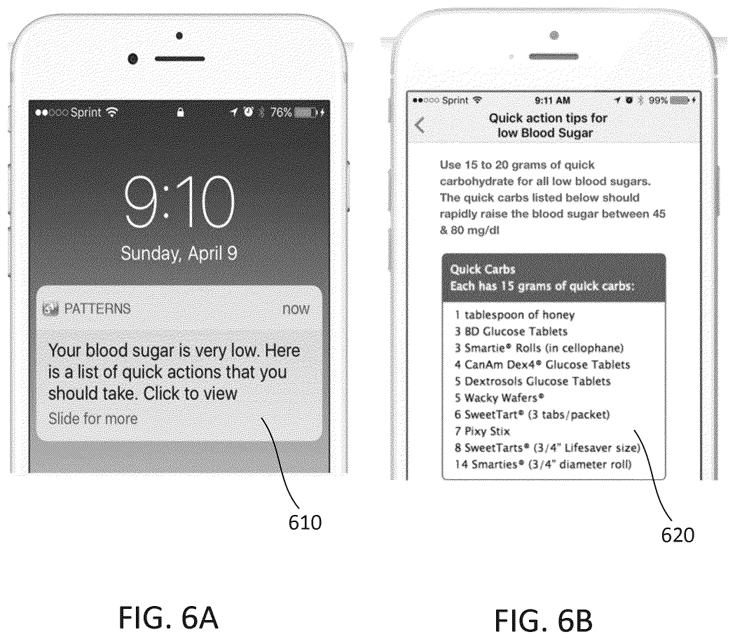

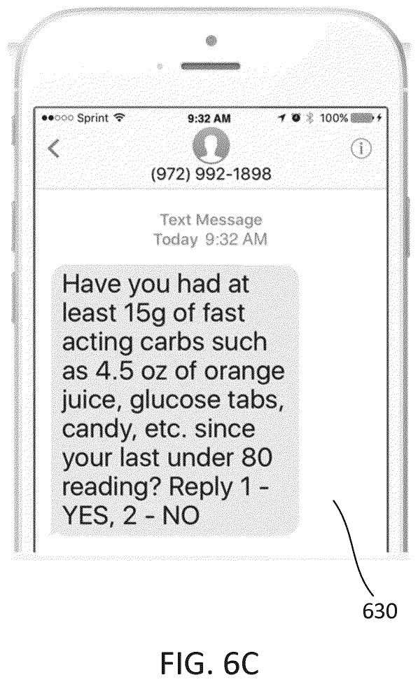

In some variations, a prompt may provide an alert or notification about a health condition or status of the patient. For example, a prompt may be generated if Current Glucose>20% of Average Glucose over a time interval or if Current Glucose<20% of Average Glucose over a time interval. It should be understood that other suitable threshold values may be used to trigger generation of a prompt. In some exemplary variations, the prompts as illustrated in FIGS. 6A-6C may be output in response to the patient's current glucose measurement exceeding historical averages. In some variations, the Average Glucose may correspond to the average glucose measurement of a set of other patients similar to the patient (e.g., same risk group, other shared characteristics).

In some variations, a prompt may encourage the patient to test more frequently, to consult a physician or to change the user's diet and suggest and/or execute device setting modifications to further those steps. For example, if a patient consistently tests at certain times throughout the day and the patient fails to test at a time previously identified as a regular testing time, the prompt may remind the patient to test and suggest setting a notification from the computing device and/or analyte measurement device (e.g., “Would you like to set a daily reminder to test at 8:00 a.m.?”). A visual, audible, and/or haptic alarm may be set to notify the patient to perform a test at a scheduled testing time. The patient may input confirmation to change the device settings as indicated in the prompt. In the variations and examples described herein, the prompt may execute a command automatically or require user input to confirm.

In some variations, if the time a patient tests are consistently outside of a prescribed or historically calculated range, a prompt may suggest setting a notification on the computing device and/or analyte measurement device to test before the prescribed or historically calculated range passes. Patient confirmation of the prompt may modify the device settings as indicated in the prompt. The prompt may be output on any computing device described herein. If the modified device settings do not increase testing compliance over time, one or more additional device setting modification prompts may be output to the patient.

In some variations, a prompt may be generated to suggest setting a notification to test and/or engage in physical activity based on trend analysis that indicates high glucose and/or inactivity around a certain time of day (e.g., “Your glucose is spiking on days when you are less active. Would you like to set an activity reminder?”). A user may input to the computing device a one button confirmation to command the computing device modify the device settings to set a recurring activity reminder based on the trend analysis.

In some variations, a prompt may be generated to suggest setting a notification to test and/or cat based on trend analysis that indicates a consistent failure to test and/or engage in physical activity around a certain time of day. In other variations, a prompt may be generated to suggest setting a notification to test if an interval between tests exceeds a predetermined threshold.

In some variations, a prompt may inform a user that a potential glucose event may occur in the future and suggest a command to schedule an appointment with their health care professional using the computing device (e.g., “Do you wish to schedule a doctor's appointment?”). The user may confirm execution of the command in the prompt (e.g., establish a voice call, schedule an appointment) by making an input to the computing device. For example, data analysis may indicate a trend of consistent high blood glucose (BG) in the morning (e.g., before breakfast) that may indicate a possible deficiency with basal insulin dosage. The prompt may suggest a command to schedule an appointment with their health care professional to discuss the data analysis. Thus, analysis and a course of action may be generated and executed with little or no patient action after completion of a measurement test. Any negative trends in the data may be identified early (e.g., before a doctor visit) and notified to the patient before the patient's health risk increases. By contrast, a typical patient would likely wait until their next regularly scheduled visit (e.g., up to 90 days or more) before discussing their glucose measurements with their health care professional. Since typical glucose meters do not calculate an average by time of day, a patient's health care professional may not even notice that a patient's glucose has been out of preferred range in the morning.

As another example, analysis of analyte measuring data and patient data may indicate that the patient has not eaten recently and has a blood sugar value of about 40 mg/dL, which is considered dangerously low under American Diabetes Association (ADA) guidelines, which defines hypoglycemia as a blood sugar level below 70 mg/dL. A prompt may be generated based on this analysis and output to the patient with a suggestion to consume carbohydrates to increase blood sugar and schedule an appointment with their health care professional (e.g., doctor). If the patient inputs to decline the suggested appointment scheduling, a follow up prompt may be output to a patient at a later time to suggest again that an appointment be scheduled. If the patient inputs to confirm the suggestion, the computing device may modify the device settings to schedule an appointment. In some variations, the appointment may be recurring. In some variations, the prompt may include observations including one or more ADA guidelines.

In some variations, one or more of the prompts may depend on the results of a determination of when a glucose event may occur. A prompt may inform a patient of the likelihood of a glucose event occurring. For example, the prompt may inform a patient that a glucose event is imminent and also suggest that the computing device establish a voice call or videoconference with a set of predetermined contacts (e.g., partner, family member, friends, social group, support group, health care professional, coach, etc.). For example, a glucose measurement below a certain range, for example less than 60 mg/dL may output a prompt notifying that the computing device will contact one or more of the patient's predetermined contacts using one or more communication methods (e.g., automated phone call, text message, e-mail, social media, message board, and the like). The predetermined contacts may be notified of one or more of the patient's glucose measurement and geolocation. In some variations, notification to the set of predetermined contacts may be performed by a remote server (220) and/or computing device (222) separate from the analyte measurement device (210), patient measurement device (212), and computing device (220). In some variations, a communication channel between the patient computing device (220) and a predetermined contact device (222) may be initiated by a third-party device (e.g., remote server (220)). In another example, a glucose measurement below 40 mg/dL may output a prompt to the patient's phone requesting confirmation that the patient is OK. In yet another example, a glucose measurement that is a predetermined amount (e.g., two standard deviations) below a patient's average glucose measurement of the last 30 days may trigger a prompt to one or more of the patient's predetermined contacts. In some variations, a patient response to a glucose event prompt may generate a prompt to a single contact such as the patient's partner while a lack of patient response to the glucose event prompt may generate additional prompts to each of the patient's contacts including a health care professional.

In some variations, a set of predetermined contacts of a patient may include a set of other patients having similar characteristics. For example, a set of patients may be classified as being in the same risk group as described in more detail herein. This set of patients may be transmitted to a patient computing device and the patient may opt to add one or more patients of the received set of patients as their support group of predetermined contacts. A risk group may be divided into subgroups by one or more additional criteria, including but not limited to age, gender, weight, race, address, activity level, health, goals, organization, employer, geographical location, health care professional, coach, and the like. The set of predetermined contacts may be limited to a small group (e.g., one, two, three, four, twelve or less). In some of these variations, when a prompt is generated due to the likelihood of a glucose event occurring, the predetermined contacts including the support group may receive the prompt. In some of these variations, the prompt may be of a positive nature to inform the support group of the patient achieving a predetermined consistency of patient compliance to encourage the group and/or create a competitive group dynamic. In some of these variations, the prompt may be limited to non-emergency prompts. In some variations, the patient may be connected to the support group through a social media platform. Over time, the members of the support group may change and/or the patient may be reclassified into a different support group based on updated data trend analysis. In some variations, the set of predetermined contacts may include a set of patients from a different risk group. For example, a patient may be paired with another patient (e.g., from a lower risk group than the patient's risk group) to form a mentor-mentee relationship. In some variations, the set of predetermined contacts may include a set of patients from the same risk group. For example, a patient may be paired with another patient of the same risk group to form a co-motivational relationship (e.g., “buddy” system, accountability partner). In some variations, a set of suggested predetermined contacts (e.g., support group) may be performed by, for example, a controller of a remote server (220) and/or computing device (222) separate from the analyte measurement device (210), patient measurement device (212), and computing device (220).

In some variations, data analysis of analyte data and patient data indicating consistent low blood glucose after lunch may suggest insulin sensitivity that is higher midday such that the patient may need less insulin around that time. A prompt may be generated to suggest that the patient contact their health care professional to discuss adjusting the patient's insulin-to-carbohydrate ratio during the day. The prompt may also suggest or automatically increase a hypoglycemic alert threshold. A more sensitive threshold may allow a patient in a potentially dangerous condition to be identified more quickly.

In some variations, the actionable prompts may be output to one or more of the patient and a set of predetermined contacts through mobile Push notifications, text messages, voice calls, e-mails, and the like. In some variations, the prompt may include information related to health, fitness, diet, lifestyle, motivation, education, promotion, and other writing that may encourage the patient to make changes and maintain consistency in improving their health with a suggestion related to the information such as sharing the information with the patient's set of predetermined contacts, archiving the prompt for future reference by the patient, and linking the patient to additional, related information. In some variations, the prompt may include a link for the patient to connect to any of a website, application, communication platform (e.g., social media network), and GUI.

An illustrative variation of a device modification process is depicted in FIG. 3B. After generating a set of prompts, a determination may be made whether health care professional (HCP) intervention is recommended (320) based on the data analysis and trends. If not (320—No), then measurement data and analysis trends may be output to the patient (322) through a computing device optionally with a device modification prompt. If intervention is recommended (320—Yes), then a determination may be made whether immediate attention is recommended (324) based on the data analysis and trends. If not (324—No), then a prompt may be output including a command to suggest an appointment be scheduled between the patient and health care professional (326). The patient may confirm or decline the prompt through input to the computing device. If immediate intervention by a health care professional is recommended (324—Yes), then the patient's health care professional may be notified and given access to the patient's data and trend analysis (328). The health care professional may be provided access to the same analysis available to the patient. In some variations, additional analysis may be generated specifically for the health care professional as well as modification options related to a patient's prescription. One or more of steps 320-328 may be at least partially performed by a controller of a remote server (220) and/or computing device (222).

A communication channel may be established between the patient and the health care professional (330) and/or a set of predetermined contacts (e.g., partner, family member, social group, coach, friend). For example, a voice call or videoconference may be established between respective computing devices of the patient and health care professional. In some variations, the health care professional may correspond to a doctor, nurse, coach, diabetes educator, dietitian, rehabilitation professional, pharmacist, emergency medical service professional, mental health professional, allied health professional, and the like.

A determination may be made whether a prompt should include a command to change device settings of either the patient computing device or health care professional computing device (332) based on the patient's data and trend analysis. If not (332—No), measurement data and analysis trends may be output to the patient (322). If the prompt should include device setting modifications (332—Yes), then a patient prompt may be output for the health care professional to review (334). One or more of steps 332-334 may be at least partially performed by a controller of a remote server (220) and/or computing device (222).

For example, the health care professional may review and select a prompt to modify a patient's prescription (e.g., modify medicine, dosing). In some variations, the patient prompt for health care professional review may comprise one or more of medical suggestions (e.g., medical information related to facts and information) and medical advice (e.g., diagnosis and/or prescribing a treatment for a medical condition). For example, the patient prompt generated for a coach and/or diabetes educator to review may be limited to medical suggestions and/or observations based on health guidelines and/or standards set by a health organization (e.g., diabetes organization, wellness organization). The prompt output to the patient may be limited to medical suggestions and not include medical advice. For example, automated coaching, as described in more detail herein, may generate patient prompts in response to a patient query based on the patient's data and trend analysis and ADA guidelines. Additionally or alternatively, the prompt may output suggested modifications to a health care professional computing device. For example, the prompt may suggest a notification be set on the health care professional computing device to follow up with the patient and/or review the patient's data on a more frequent basis.

Moreover, the prompt provided to a health care professional may differ in tone from the prompt provided to a patient. The patient prompt may be softened (e.g., neutral, non-judgmental, encouraging, simplified) to increase patient compliance and reduce stress and discouragement associated with disease management. In contrast, the health care professional prompt may be more succinct, scientific, and clinical. For example, a patient prompt may be presented in an encouraging tone; “Hi John, we've detected that your glucose has been trending very high over the last week. Would you like help on how to try and lower your glucose?” On the other hand, a health care professional prompt may be more direct; “Patient John S. has had 15 hyperglycemic events in the last week. John S's A1c is currently at 9.1 and he is at risk for serious complications. It may be beneficial to intervene with a change in prescription, increased test frequency, and/or suggestion to make dietary changes.”

The health care professional may input a command to execute the prompt for the patient (336). For example, the health care professional may select the prompt to be output to the patient from a list of generated prompts output in step 334. Additionally or alternatively, the health care professional may execute a prompt to modify device settings of the health care professional device. The device settings may be modified (338) for one or more patient computing devices and health care professional devices. One or more of steps 336 and 338 may be at least partially performed by a controller of a remote server (220) and/or computing device (222).

In some variations, a patient at their own discretion may proactively establish a communication channel for a session with a coach or health care professional (e.g., nurse, diabetes educator) and/or automated (e.g., guided) coach to receive at least one of suggestions, observations, and support. For example, the patient may have a set of one or more questions regarding their trend analysis and/or may desire emotional and/or logistical support in implementing behavior change. In some variations, a patient may input a query using a computing device that may include a request to communicate with a coach and/or health care professional. The query may be input in any suitable manner including text input via keyboard, selection from a displayed list of selectable queries, speech or dictation, etc. In some variations, the patient may have limited access to a coach and/or health care professional. For example, the patient's access may be limited in duration (e.g., a thirty-minute call, videoconference, or text or chat session), frequency (e.g., predetermined number of sessions per month, week, or day), or duration of access (e.g., unlimited number of sessions during a predetermined period of time). In some variations, the patient may have unlimited access to a coach and/or health care professional. The nature of the patient's access may be based at least in part on, for example, a subscription service and/or the patient's risk classification.

In some variations, the patient may be asked to select a topic from a predetermined list of topics such as nutrition, sleep, mental health, and the like. A communication channel may then be established between the patient and a suitable coach or health care professional. In some variations, a health care professional computing device may output one or more of the patient's query, analyte data, patient data, and trend analysis for the session.

For example, a patient interested in improving their dict may input a nutrition question to a GUI to initiate and establish a communication channel with a dietician. In some of these variations, the communication may be limited to suggestions and observations and not include medical advice. For example, the dietitian may be provided a predetermined set of suggestions and observations in line with accepted guidelines that are to be followed in conversation with the patient.

In some variations of automated coaching, the patient may be asked to select a topic (e.g., question) from a predetermined list of common topics. The patient may be subsequently asked to answer a set of questions related to the topic. One or more automated prompts may be generated based on the topic, patient input, and trend analysis to provide suggestions and/or observations to help the patient. For example, a patient may be presented a list of topics related to improving their wellness indicator by, for example, incorporating an exercise routine into their lifestyle. The patient may select this topic and then be presented for selection a set of fitness goals (e.g., lose 10 pounds, run a 5K race, etc.), activities (e.g., walking, cycling, running, swimming, etc.) that interest them, and availability (e.g., during lunch, after work). Based on the patient input and previously acquired patient activity data, a prompt may be automatically generated that provides contact information for a swim club and/or gym within 5 miles of the patient's place of work. The prompt may further prompt the patient to call and/or navigate to the website of the club and/or gym. The prompt may also display the ADA recommendations for physical activity (e.g., 30 minutes of moderate-to-vigorous intensity aerobic exercise at least 5 days a week or a total of 150 minutes per week). As another example, the patient may be provided a list of “Frequently Asked Questions” such that the patient may select a query or topic of interest to obtain more information.

### Managing a Patient Population

A single health care professional is commonly responsible for the treatment of hundreds or even thousands of patients with chronic conditions such as diabetes. Current solutions require health care professionals to manually sort through glucose testing data and medical records to attempt to identify which patients to prioritize for intervention. The review of data is tedious and time consuming, and due to dispersed and unintegrated sets of data, a health care professional may not even have access to relevant data from any available measurement device (e.g., activity tracker, scale, blood pressure monitor, and the like). Conventionally, a significant amount of time and money is spent on the collection, review and manual analysis of data. FIG. 3C depicts an illustrative variation of a method of managing a patient population where device data corresponding to a plurality of patients may be leveraged to prioritize patient care, as well as generate more relevant trends and effective prompts.

Prioritization of a patient docket may aid in managing a patient population and improving patient care. For example, by automatically collecting, analyzing, organizing, and presenting measurement data to a health care professional, a set of patients may be prioritized for intervention with little to any input from a health care professional. Furthermore, the health care professional may be provided a prompt suggesting actions they may take with respect to patient treatment for one or more patient subsets. In some variations, patients classified as high risk may be given highest priority. In other variations, patients who may be quickly addressed may be given higher priority.

In some variations, a database and/or computing device may store analyte data and patient data for a plurality of patients from a plurality of measurement devices. The data of each patient may be anonymized to ensure privacy. Patient trends may be generated by analyzing data trends of each patient against each other (340). Patient trends may be used to identify patients on a negative trend that may benefit from additional support from a health care professional. Patients may be classified into groups (e.g., risk levels) using the patient trends (342). For example, patients may be classified into different groups based on similarity in one or more data trends, analyte data, age, gender, medical history, weight, race, activity level, health, goals, organization (e.g., company, employer, office, club, etc.), combinations thereof, and the like. As another example, the classified groups may be used to generate patient support groups as described in more detail herein.

In some variations, prompts outputted to a plurality of patients may be compared against patient outcomes to determine the relative effectiveness of the prompts at making positive changes in a patient's condition. Prompt trends may be generated by analyzing data trends for each patient against the prompts output to the patients (344). In some variations, analysis of prompt trends may be performed by a remote server (220) and/or computing device (222) separate from the analyte measurement device (210), patient measurement device (212), and computing device (220). Data analysis of data trends against the prompts may identify useful trends including which prompts are most effective at increasing patient compliance and helping patients improve their condition over time. A set of prompts may be generated for the patient based on the prompt trends (346). Over time, less effective prompts may be output to patients with less frequency while more effective prompts may be output with greater frequency. For example, prompt trend analysis may indicate that prompts written in a formal tone with no graphics are less likely to correlate with patient improvement than prompts written in a conversational style and accompanied by a video. As another example, prompt trend analysis may indicate that the effectiveness of a given prompt may differ between patients in different age groups. Thereafter, the more effective prompts may be outputted at a higher frequency. The prompt may be output to the patient (348) on a computing device as described herein. The prompt may include any of the generated trends and include a suggestion to modify device settings as described herein. The patient may input a response to the prompt (350) such as to confirm or decline the suggested device modification in the prompt.

As another example, a set of similar patients (e.g., having high blood glucose in early afternoon), may receive one of three prompts: adjust insulin to carbohydrate ratio; go for an afternoon walk; swap out a carbohydrate portion for a healthy fat at lunch. Data may be generated following patient reception of the prompt. Data analysis may then be performed to generate patient trends corresponding to each of these prompts. For example, a health factor such as an estimated A1c (a measure of glucose over time) may be analyzed against the prompts given to each patient to determine the effectiveness of each prompt in changing a trend. As a result of the analysis, the frequency of output of the three prompts may be modified (e.g., the most effective prompt may be output more often than the least effective prompt). Additional prompts may be added and others deleted based on the prompt analysis. The set of prompts may be modified at predetermined intervals based on one or more of time (e.g., monthly, quarterly), number of outputted prompts, combinations thereof, and the like.

In other variations, prompts provided to a plurality of patients may include observations such as educational information (e.g., health-related article, news, etc.), motivational material (e.g., messaging), and/or other kinds of suitable information to promote better health. Such prompts may be specific or targeted to a set of patients based on one or more shared characteristics (e.g., health characteristic, characteristic of belonging to a shared, common organization, etc.). For example, a prompt may include a health-related article informing the plurality of patients about creative ways to insert exercise into a workday, a notification about expected conditions at a local outdoor walking trail, etc. Similar to that described above, a plurality of patients may, for example, be contacted substantially concurrently via a single such prompt. In some variations, the set of prompts including device modifications, observations, and information may be configured and stored by a remote server (220) and/or computing device (222) separate from the analyte measurement device (210), patient measurement device (212), and computing device (220).

Although methods for managing a patient population are described primarily with respect to a health care professional managing a patient population, other variations of the method may additionally or alternatively including providing other kinds of users with patient and/or patient population information and allowing such users to provide prompts and other information. For example, a coach, mentor, administrator (e.g., of an organization) or other user may access patient data (data of individual patients or aggregate data of sets of patients), such as through a web portal or through a user computing device (e.g., with a mobile application) and communicate with individual patients and/or sets of multiple patients. In some variations, an organization (e.g., employer) may have access to an anonymized set of patient data and analysis received from a database (e.g., remote server, network). For example, one or more of a population analytics, stratification analysis, and trend analysis may be provided to an organization to allow tracking of patient engagement, compliance, and improvement on a global basis for a particular subpopulation.

FIG. 4 is a set of illustrative variations of a graphical user interface (GUI). An analyte measurement device (410) may automatically connect (412) to a cloud database to transmit generated analyte measurement data (402). The data may be processed and analyzed (414) on a remote computing device (e.g., remote server) and the results output to a patient's smartphone through a set of GUIs. The GUIs permit a patient to view their blood glucose data and analysis via any of a mobile application and/or website (404). A first GUI (420) may include a chart of a patient's glucose measurements over time (e.g., over a 7 day period) that may allow a patient to easily visualize and understand their glucose measurements. For example, the first GUI (420) may selectively display daily, weekly, and monthly glucose reports stored and/or retrieved from a cloud database. A second GUI (430) may include a prompt (432) that may provide context to the data, charts, and/or analysis presented to the patient. For example, the prompt (432) may include encouragement and positive reinforcement to the patient for maintaining their target goals. Patient data (434) including exercise and meal data may also be graphically displayed in the second GUI (430) or any of the GUIs. A third GUI (440) may include a display of analysis of one or more trends including glucose trends (442) and meal trends (444). For example, a color coded bar graph may illustrate how often the patient's blood glucose measurements fall within a target range. The target glucose analysis in the third GUI (440) may be modified to show high and low glucose patterns and results within a desired glucose range.

In some variations, the patient may designate a set of predetermined contacts (e.g., care circle, sharing circle) that may have access to the patient's data. The fourth GUI (450) may include display of settings for designating a predetermined contact (452) in the care circle and include different methods of contacting the contact such as phone and e-mail. One or more contacts may be designated as an emergency contact that may be automatically notified in the event that the patient's data and/or analysis indicate a potentially dangerous situation. For example, a patient computing device (e.g., smartphone) may transmit an emergency alert to one or more predetermined contacts in the event that a patient's glucose exceeds a predetermined threshold. The alert may include one or more of the glucose measurements, trend analysis, and location of the patient as estimated through using the patient's smartphone GPS functionality.

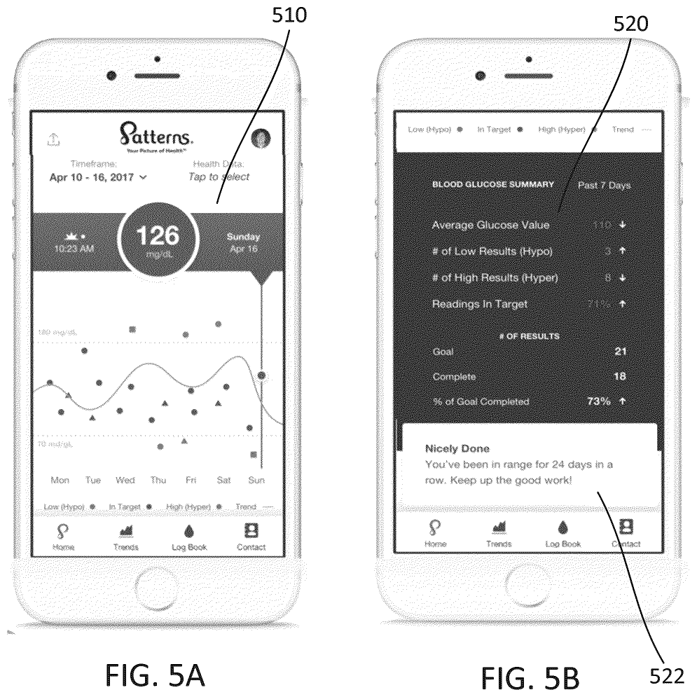

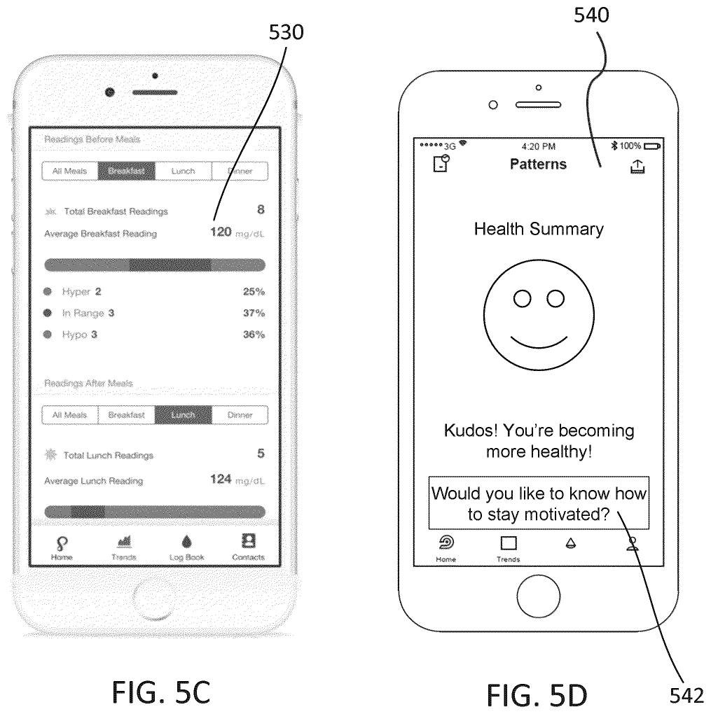

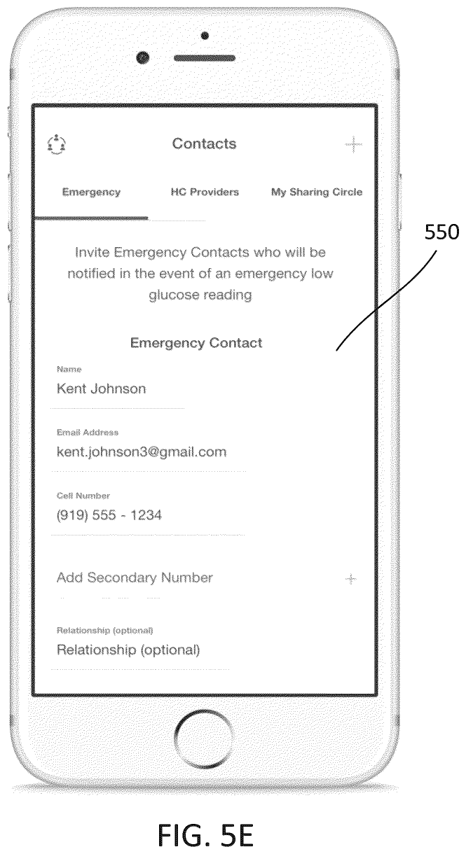

FIGS. 5A-5E is another set of illustrative variations of a GUI. In some variations, one or more of the GUIs may be output on a patient computing device (220) and may use data received from a remote computing device (e.g., remote server (250), cloud database (240)) as described in detail with respect to FIG. 2. FIG. 5A depicts a fifth GUI (510) (e.g., glucose dashboard) including a chart of a patient's glucose measurements over time (e.g., over a 7 day period) that may allow a patient to visualize and understand their glucose measurements. The plotted glucose measurements may be color coded to indicate measurements within different predetermined ranges (e.g., low, in target, high). A trend line may be overlaid on the chart. For example, a user may select one or more timeframes (e.g., daily, weekly, monthly) to generate corresponding glucose repots stored and/or retrieved from a cloud database. In some variations, the time, date, and/or value of the patient's last glucose measurement may be displayed prominently by the fifth GUI (510).

FIG. 5B depicts a sixth GUI (520) (e.g., trend indicator) providing a blood glucose summary of a set of metrics for a predetermined interval of time (e.g. over a 7 day period). Each metric may include an indicator (e.g., arrow pointing up, arrow pointing down) of how that metric is trending. For example, as indicated in FIG. 5B, the three hypoglycemic events over the last seven days is an increase over the preceding seven days, while the eight hyper hyperglycemic events over the last seven days is a decrease over the preceding seven days. The sixth GUI (520) may include a prompt (522) to provide context to the data and analysis presented to the user. For example, the prompt (522) may include encouragement and positive reinforcement to the patient for maintaining their target goals over an extended period of time. In some variations, a user may select the prompt (522) to receive additional information and/or modify device settings. For example, a set of poor trends may generate a prompt asking if the patient would like to schedule an appointment with their doctor, initiate a live coaching session, or review educational materials.

FIG. 5C depicts a seventh GUI (530) (e.g., meal summary) providing a set of meal trends. For example, a color-coded bar graph may illustrate how often the patient's blood glucose measurements fall within a target range for one or more meals (e.g., breakfast, lunch, dinner, all meals). High, low, and in range glucose patterns may be displayed.

FIG. 5D depicts an eighth GUI (540) (including, e.g., a wellness indicator) providing a summary (e.g., smiley face) of the patient's overall health based on a plurality of metrics including analyte data and patient data as described in detail herein. Similar to that described above, the wellness indicator may provide a simplified metric to help users more easily or quickly gauge the patient's overall health. The wellness indicator may be a simplified indicator of general overall health that considers and aggregates a set of health characteristics. The eighth GUI (540) may optionally include a prompt (542) providing an actionable suggestion of how the patient may increase their wellness indicator and/or observations on how the wellness indicator is derived. The prompt (542) may include observations that may provide additional context to the wellness indicator.

FIG. 5E depicts a ninth GUI (550) where a user may designate a set of one or more predetermined contacts (e.g., emergency, health care providers, sharing circle, etc.). The ninth GUI (550) may include display of settings for designating a predetermined contact in one or more groups and include different methods for contacting the contact such as phone and e-mail. One or more contacts may be designated as an emergency contact that may be automatically notified in the event that the patient's data and/or analysis indicate a potentially dangerous situation. For example, a patient computing device (e.g., smartphone) may transmit an emergency alert to one or more predetermined contacts in the event that a patient's glucose exceeds a predetermined threshold. The alert may include one or more of the glucose measurements, trend analysis, and location of the patient as estimated through using the patient's smartphone GPS functionality.

FIG. 6A-6C is another set of illustrative variations of a GUI. In some variations, one or more of the GUIs may be output on a patient computing device (220) and may use data received from a remote computing device (e.g., remote server (250), cloud database (240)) as described in detail with respect to FIG. 2. FIG. 6A depicts a tenth GUI (610) providing an actionable prompt providing a suggestion to modify patient behavior in response to analysis of a patient's analyte data and patient data. For example, the tenth GUI (610) includes display of a Push Notification Alert that suggests a user take action in response to the patient's low blood sugar. The user may input a command (e.g., swipe motion) to transition to an eleventh GUI (620) (e.g., quick action tips) depicted in FIG. 6B. In some variations, the eleventh GUI (620) may comprise one or more of text, audio, video, haptic feedback, etc. In some variations, the user may confirm having received and/or followed the suggestions and/or observations presented in the eleventh GUI (620).

FIG. 6C depicts a twelfth GUI (630) providing a confirmation prompt at a predetermined interval of time. For example, the prompt may comprise a text message transmitted to the patient's phone number a predetermined period of time (e.g., 30 minutes) after receiving the initial low blood sugar prompt. The confirmation prompt may ask the user to transmit a text message response indicating whether the patient followed the suggestion indicated in the prompt. As more time passes without user response, the frequency and urgency of the prompts may increase. For example, if no response is received within a predetermined period of time, additional text message prompts may be transmitted at predetermined intervals of time and/or a set of predetermined contacts may be notified and informed of the situation. In some variations, configuration of the prompts may be performed by the patient's health care professional and/or the patient.

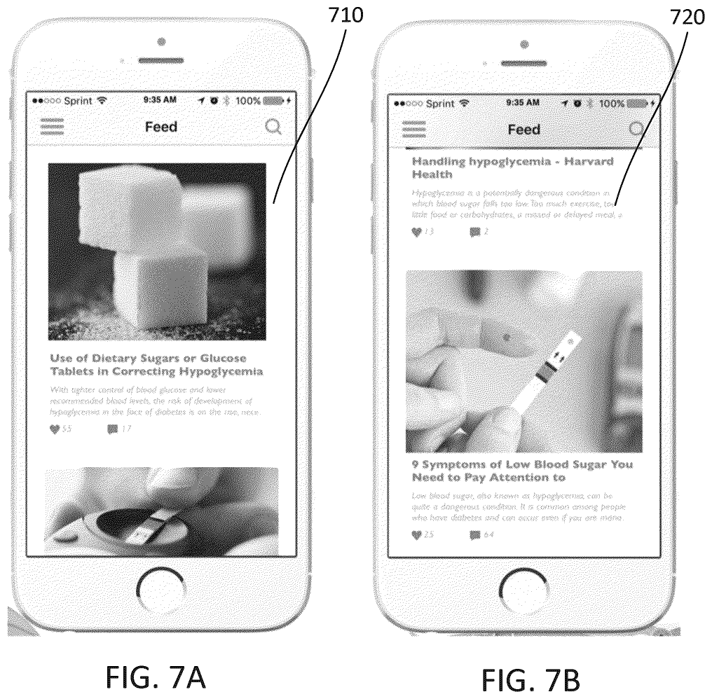

FIGS. 7A-7B is another set of illustrative variations of a GUI. In some variations, one or more of the GUIs may be output on a patient computing device (220) and may use data received from a remote computing device (e.g., remote server (250), cloud database (240)) as described in detail with respect to FIG. 2. FIG. 7A depicts a thirteenth GUI (710) (e.g., news or information feed) and FIG. 7B depicts a fourteenth GUI (720) providing a set of educational and/or motivational material for a patient based on analysis of the patient's analyte data and patient data. For example, a patient experiencing an increase in the number of low blood glucose events in successive weeks may trigger generation of a prompt including health articles related to hypoglycemia. In some variations, the user may be asked to confirm they have read the articles and/or asked to answer questions related to the article's contents.

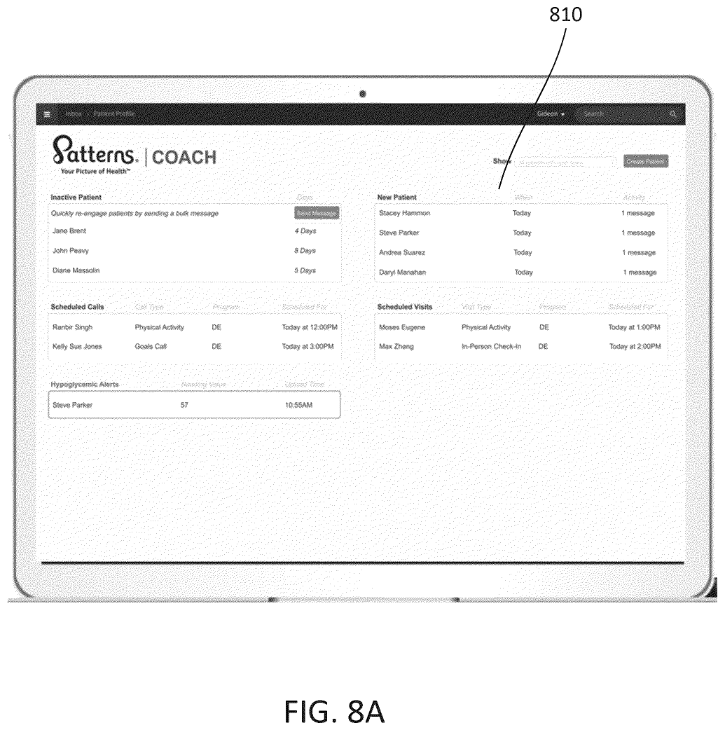

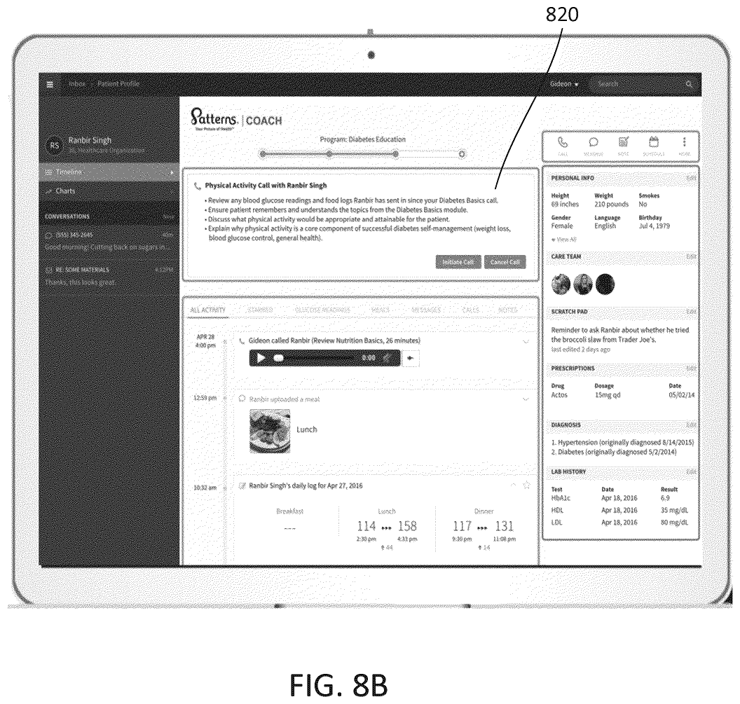

FIGS. 8A-8B is another set of illustrative variations of a GUI. In some variations, one or more of the GUIs may be output on a health care professional computing device (222) and may usc data received from a patient computing device (220) and/or remote computing device (e.g., remote server (250), cloud database (240)) as described in detail with respect to FIG. 2. The variations of a GUI depicted in FIGS. 8A-8B may, for example, be accessible via a web portal.

FIG. 8A depicts a fifteenth GUI (810) providing a patient docket of a coach and/or health care professional's caseload. Analysis of a set of patient and analyte data may be used to group the patients into subsets (e.g., high risk, low risk, urgent, inactive, new patient, scheduled, etc.). For example, inactive patients who have not tested within a predetermined interval of time (e.g., 3 or more days) may be grouped together such that the coach and/or health care professional may send a bulk message to the group to encourage compliance and/or engagement. High risk patients such as those experiencing a hypoglycemic event in real-time may also be presented to the coach and/or health care professional. The coach and/or health care professional may receive a prompt to establish a communication channel with these patients based on predetermined thresholds.

FIG. 8B depicts a sixteenth GUI (820) providing patient and analyte data of a patient to a coach and/or health care professional. The sixteenth GUI (820) may include patient data including one or more of general health information (e.g., height, weight, gender, age, etc.), prescription information, diagnosis, testing history, charts, timeline, contact information, employer information, insurance information, program information (e.g., diabetes education, motivation, compliance), and notes, and the like. In some variations, the coach and/or health care professional may be provided a prompt to establish a communication channel with the patient (e.g., voice call, video conference) and/or a suggested agenda for discussion with the patient. In some variations, the coach and/or health care professional may be provided a more detailed view of the patient and analyte data than the patient. In some variations, the coach and/or health care professional may transmit information (e.g., images, video, text) to display on the patient computing device while the communication channel with the patient is active. In some variations, one or more prompts presented to a coach may guide the information provided by the coach to the patient.

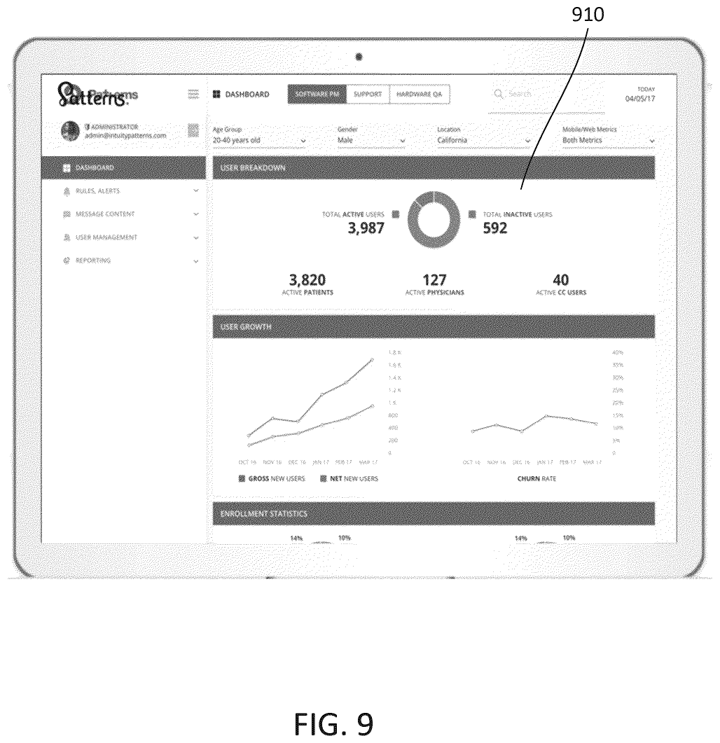

FIG. 9 is another illustrative variation of a GUI. In some variations, one or more of the GUIs may be output on a computing device and may use data received from a patient computing device (220) and/or remote computing device (e.g., remote server (250), cloud database (240)) as described in detail with respect to FIG. 2. The variation of a GUI depicted in FIG. 9 may, for example, be accessible via a web portal.

FIG. 9 depicts a seventeenth GUI (910) (e.g., organization dashboard) providing trend and analysis data for a set of patients of an organization (e.g., employer). In some variations, an organization that has enrolled the set of patients may have access to an anonymized set of patient data and analysis of all of the patients. For example, one or more of a population analytics, stratification analysis, and trend analysis may be provided to an organization (e.g., administrator of the organization) to allow tracking of patient engagement, compliance, and improvement. In FIG. 9, the set of patients may be filtered by one or more of age, gender, location, computing device, etc. Patient analysis may include user growth, enrollment, and/or demographic information. In some variations, a set of prompt settings including rules and alerts may be configured by the organization.

The specific examples and descriptions herein are exemplary in nature and variations may be developed by those skilled in the art based on the material taught herein without departing from the scope of the present invention, which is limited only by the attached claims.

1. A method of monitoring a chronic condition of a patient, comprising:
receiving analyte data generated by an analyte measurement device and patient data generated by a patient measurement device;
generating one or more data trends by analyzing the analyte data against the patient data using a computing device comprising a processor and memory; and
modifying device settings of one or more of the analyte measurement device and the computing device in response to one or more of the data trends.

2. The method of claim 1, further comprising outputting at least one prompt to modify patient behavior in response to one or more of the data trends.

3. The method of claim 2, wherein the prompt may comprise encouragement to comply with one or more of a testing, diet, and exercise regimen.

4. The method of claim 1, further comprising outputting at least one prompt to modify the device settings in response to one or more of the data trends.

5. (canceled)

6. The method of claim 1, wherein modifying the device settings comprises modifying one or more of frequency, timing, and content of patient notification.

7. The method of claim 1, further comprising notifying a set of one or more predetermined contacts based on a characteristic of the one or more data trends.

8.-12. (canceled)

13. The method of claim 1, further comprising outputting at least one prompt to modify health care professional device settings in response to one or more of the data trends.

14. The method of claim 1, further comprising establishing a communication channel between the computing device and a health care professional device in response to one or more of the data trends being a high risk condition.

15. The method of claim 14, further comprising receiving the analyte data, the patient data, and the one or more data trends at the health care professional device, and outputting a prompt to modify patient behavior and the device settings at the health care provider device.

16.-26. (canceled)

27. The method of claim 1, further comprising receiving a patient query and outputting at least one prompt to modify at least one of patient behavior and device settings in response to one or more of the data trends.

28. The method of claim 1, further comprising transferring the analyte data from the analyte measurement device to the computing device at predetermined intervals.

29. The method of claim 1, further comprising outputting the one or more data trends using the computing device.

30. A method of managing a patient population, comprising:
receiving analyte data generated by a plurality of analyte measurement devices and patient data generated by a plurality of patient measurement devices, the analyte data and the patient data corresponding to a plurality of patients;
generating one or more data trends by analyzing the analyte data against the patient data for the plurality of patients using a computing device comprising a processor and memory;
classifying at least a portion of the plurality of patients based on risk using the one or more data trends; and
selecting a patient for treatment from a health care professional based on the patient classification.

31. The method of claim 30, further comprising generating an intervention plan for the selected patient using the one or more data trends, and outputting the intervention plan to the health care professional.

32. The method of claim 30, further comprising generating a set of one or more prompts to modify the device settings of a patient computing device based on the risk.

33. The method of claim 30, further comprising generating one or more patient trends by analyzing the one or more data trends among the plurality of patients.

34. The method of claim 33, further comprising:
generating one or more prompt trends by analyzing the one or more patient trends against the set of prompts;
classifying the prompts based on prompt effectiveness, wherein effectiveness is based on one or more of the prompt trends; and
outputting a selected prompt from the set of prompts to the patient computing device based on the effectiveness of the selected prompt.

35. The method of claim 30, further comprising generating a support group for the selected patient based at least in part on the patient classification, wherein the support group comprises at least one other patient from the plurality of patients.

36. The method of claim 35, further comprising generating a prompt for the selected patient to join the support group.

37. A device, comprising:
a transceiver configured to receive analyte data generated by an analyte measurement device and patient data generated by a patient measurement device; and
a controller coupled to the transceiver, the controller comprising a processor and a memory, and the controller configured to:
generate one or more data trends by analyzing the analyte data against the patient data;
generate a prompt to modify patient computing device settings in response to one or more of the data trends; and
output the prompt to a patient computing device using the transceiver.
